(19) United States
(12) Patent Application Publication
(10) Pub. No.: US 2026/0125716 A1
(43) Pub. Date: May 7, 2026

(54) Optimised Fermentation of Anaerobic Bacteria

(71) Applicant: Again Bio ApS, Copenhagen (DK)

(72) Inventors: Andreea-Cristina Dobrescu, Copenhagen (DK); Carolina Santos Giordani Benevenuti, Copenhagen (DK); Stephanie REDL, Copenhagen (DK); Torbjørn Ølshøj Jensen, Stenloese (DK)

(73) Assignee: Again Bio ApS, Copenhagen (DK)

(21) Appl. No.: 19/118,040

(22) PCT Filed: Oct. 6, 2023
(86) PCT No.: PCT/EP2023/077790

§ 371 Date: Apr. 3, 2025

(30) Foreign Application Priority Data
Oct. 6, 2022 (EP) 22199963.4

(51) Int. Cl. C12P 7/54; C12N 1/20; C12R 1/01

(52) U.S. Cl. CPC C12P 7/54; C12N 1/20; C12R 2001/01

(57) ABSTRACT

The present invention provides a method for increasing the growth efficiency in an anaerobic microbial fermentation of a microorganism capable of converting CO₂, CO or a mixture thereof into at least one organic compound, the method comprising growing the microorganisms in a cultivation medium comprising selenium in a concentration which is at least 1.2 μM.

### TECHNICAL FIELD OF THE INVENTION

The present invention relates to the fields of biotechnology, microbiology, fermentations and microbial growth and conversion of carbon dioxide or carbon monoxide into organic compounds.

### BACKGROUND OF THE INVENTION

Carbon emission gasses such as carbon dioxide (CO₂) and carbon monoxide (CO) form during many industrial processes, mainly manufacturing, raw material mining, energy and transportation. CO₂ and CO have traditionally been emitted to the atmosphere as rather harmless compounds entering the global carbon cycle while environmental and health safety focus was on limiting emissions of other gasses, including toxic gasses, such as NOx, SO₂, methane etc. Due to increasing global population, the ongoing global warming and climate changes there is an urgent need for industrial processes which are more circular in nature, i.e. leaving little footprints and not causing large emissions disturbing global balances. No doubt, this undertaking will require many different efforts in order to timely intervene in the ongoing developments which are very fast compared to the rate of fundamentally changing the industrial processes which support man kind and our present population. Examples being substituting combustion of fossil fuels for electricity made from wind, solar, water and nuclear power, and being minimizing the emission of CO₂ and CO from industrial processes by alternative processes or by capturing the CO₂ and CO.

The capture of CO₂ and CO from industrial processes may fundamentally be carried out in two different ways. Firstly, CO₂ may be collected for the industrial process and stored so that it does not enter the atmosphere. Attempts have been made to pump the CO₂ into underground cavities such as in areas where oil and gas exploration have taken place. There are also large scale processes where CO₂ is liquefied and transported to a suitable site for long term storage. However, the concept of storing CO₂ does point to a less than optimal solution from a sustainability point of view. Secondly, CO₂ may be collected from the industrial process where it is produced and then used in another process where it is converted into some carbon-containing product which has commercial value.

WO 98/37179 discloses chemically defined media for the fermentative production of valuable compounds on an industrial scale.

WO 2010/064932 discloses optimized fermentation media for the production of alcohols by microbial fermentation of substrates comprising CO.

US 2015/0024449 discloses a process and medium for fermenting syngas wherein the selenium levels in biomass are reduced.

Redl et al., Frontiers in Microbiology (2020), Vol 10, pp 1-15 discloses a medium for the cultivation of species of Moorella.

Hence, there is a great need for new effective processes for converting CO₂ or CO from emissions from industrial processes into products having commercial value, thus serving dual purposes of eliminating CO₂ or CO from emissions and manufacturing a product which can ensure the process to be commercially viable.

### SUMMARY OF THE INVENTION

It is an object of the present invention to provide processes for the conversion of CO₂ and CO from emissions into an organic compound. It is in particular an object of the present invention to provide an improved fermentation medium useful in a microbial process that converts CO₂ or CO into at least one organic compound. It is a further object of the present invention to provide an improved fermentation medium which facilitates a cost-effective process, e.g. low cost of medium and low cooling requirements. It is also an object of the present invention to overcome some of the disadvantages of the processes known in the art.

The inventors have surprisingly found that a medium having a high concentrations of selenium and molybdenum facilitates a high growth rate and high biomass yields of an acetogenic microorganism. The inventors has also found that the nickel concentration and the magnesium concentration in contrast to some prior teachings have novel and optimal intervals with respect to growth rate and yield of an acetogenic microorganism

In a first aspect the present invention provides a method for increasing the growth efficiency in an anaerobic microbial fermentation of a microorganism capable of converting CO₂, CO or a mixture thereof into at least one organic compound, the method comprising growing the microorganisms in a cultivation medium comprising selenium in a concentration which is at least 1.2 μM.

In a second aspect the present invention provides a method for increasing the growth efficiency in an anaerobic microbial fermentation of a microorganism converting CO₂, CO or a mixture thereof into at least one organic compound, the method comprising growing the microorganisms in a cultivation medium comprising nickel in a concentration which is in the range from 3.0 μM to 8.5 μM.

In a third aspect the present invention provides a method for increasing the growth efficiency in an anaerobic microbial fermentation of a microorganism converting CO₂, CO or a mixture thereof into at least one organic compound, the method comprising growing the microorganisms in a cultivation medium comprising molybdenum in a concentration which is at least 1.5 μM.

In a fourth aspect the present invention provides a method for switching from a phase of mainly biomass growth to a phase of mainly product formation for an anaerobic microbial fermentation of a microorganism capable of converting CO₂, CO or a mixture thereof into at least one organic compound, wherein said product is said at least one organic compound and said method comprising growing the microorganisms in a cultivation medium wherein the concentrations of molybdenum and nickel remains substantially constant during the switch, and the concentrations of selenium and magnesium both increases 5-15 fold during the switch.

In a fifth aspect the present invention provides the use of the method as defined in any of the first to fourth aspects for the industrial manufacture of at least one organic compound from CO₂, CO or a mixture thereof.

In an embodiment of the invention the microorganism is an acetogen, i.e. a microorganism having a metabolism converting CO₂ (with H₂) or CO into acetyl-coenzyme-A.

In another embodiment the microorganism is selected from the group consisting of Clostridium, Moorella, Thermoanaerobacter, Thermoanaerobacterium, Acetogenium, Acetobacterium, Acetoanaerobium, Butyribacterium, Eubacterium, Pyrococcus, Desulfobacterium and Carboxydothermus.

The invention is further summarized in the following list of items:

1. A method for increasing the growth efficiency in an anaerobic microbial fermentation of a microorganism capable of converting CO₂, CO or a mixture thereof into at least one organic compound, the method comprising growing the microorganisms in a cultivation medium comprising selenium in a concentration which is at least 1.2 μM.

2. A method for increasing the growth efficiency in an anaerobic microbial fermentation of a microorganism converting CO₂, CO or a mixture thereof into at least one organic compound, the method comprising growing the microorganisms in a cultivation medium comprising nickel in a concentration which is in the range from 3.0 μM to 8.5 μM.

3. A method for increasing the growth efficiency in an anaerobic microbial fermentation of a microorganism converting CO₂, CO or a mixture thereof into at least one organic compound, the method comprising growing the microorganisms in a cultivation medium comprising molybdenum in a concentration which is at least 1.5 μM.

4. The method according to any of the preceding items, wherein said cultivation medium comprises selenium in a concentration which is at least 1.3 μM, at least 2 μM, at least 3 μM, or at least 4 μM.

5. The method according to any of the preceding items, wherein said cultivation medium comprises selenium in a concentration which is in the range from 2.0 μM to 8 μM, in the range from 3.0 μM to 7 μM, in the range from 4 μM to 6 μM or in the range from 4.4 μM to 5.6 μM.

6. The method according to any of the preceding items, wherein said cultivation medium comprises selenium in a concentration which is in the range from 4.4 μM to 5.6 μM.

7. The method according to any of the preceding items, wherein said microorganism grows on a hexose as the main carbon source, such as fructose or glucose.

8. The method according to any of the preceding items, wherein said cultivation medium comprises selenium in a concentration which is at least 5.0 μM, at least 10 μM, at least 20 μM, or at least 30 μM.

9. The method according to any of the preceding items, wherein said cultivation medium comprises selenium in a concentration which is in the range from 5.0 μM to 70 μM, in the range from 10 μM to 60 μM, in the range from 20 μM to 50 μM or in the range from 30 μM to 50 μM.

10. The method according to any of items 1-6 and 8-9, wherein said microorganism grows on a gas as the main carbon source, such as CO₂ or CO.

11. The method according to any of the preceding items, wherein said cultivation medium comprises nickel in a concentration which is in the range from 4.0 μM to 8.0 μM, in the range from 5.0 μM to 7.0 μM or in the range from 5.5 μM to 7.0 μM.

12. The method according to any of the preceding items, wherein said cultivation medium comprises molybdenum in a concentration which is at least 1.7 μM, at least 2.0 μM, or at least 2.2 μM.

13. The method according to any of the preceding items, wherein said cultivation medium comprises molybdenum in a concentration which is in the range from 1.5 μM to 20 μM, in the range from 1.5 μM to 10 μM or in the range from 2.0 μM to 5 μM.

14. The method according to any of the preceding items, wherein the concentration of magnesium in said cultivation medium is in the range from 0.05 mM to 40 mM.

15. The method according to item 14, wherein said microorganism grows on a hexose as the main carbon source and the concentration of magnesium in said cultivation medium is in the range from 0.05 mM to 1.3 mM, from 0.10 mM to 0.8 mM, or from 0.15 mM to 0.30 mM.

16. The method according to item 14, wherein said microorganism grows on a gas such as CO₂ or CO as the main carbon source and the concentration of magnesium in said cultivation medium is in the range from 10 mM to 40 mM, from 15 mM to 35 mM, or from 18 mM to 27 mM.

17. The method according to any of the preceding items, wherein said microorganism is capable of converting CO₂ into at least one organic compound.

18. The method according to any of the preceding items, wherein said microorganism is capable of converting CO into at least one organic compound.

19. The method according to any of the preceding items, wherein said at least one organic compound is a C₁₋₆-compound.

20. The method according to item 9a, wherein said C₁₋₆-compound is a C₁₋₆-alcohol, a C₁₋₆-carboxylic acid or a C₁₋₆-ketone.

21. The method according to any of the preceding items, wherein said microorganism is an acetogen.

22. The method according to any of the preceding items, wherein said microorganism is selected from the group consisting of Clostridium, Moorella, Thermoanaerobacter, Thermoanaerobacterium, Acetogenium, Acetobacterium, Acetoanaerobium, Butyribacterium, Eubacterium, Pyrococcus, Desulfobacterium and Carboxydothermus.

23. The method according to item 22, wherein said microorganism is Moorella or Thermoanaerobacter.

24. The method according to item 23, wherein said microorganism is Moorella thermoacetica or Moorella thermoautotrophica.

25. The method according to any of the preceding items, wherein said at least one organic compound comprises acetate.

26. The method according to any of the preceding items, wherein said at least one organic compound comprises ethanol.

27. The method according to any of the preceding items, wherein said anaerobic microbial fermentation is a biomass propagation step.

28. The method according to any of items 1-9 and 11-27, wherein said cultivation medium comprises a hexose as the main carbon source.

29. The method according to item 28, wherein said hexose is fructose or glucose.

30. The method according to any of items 1-26, wherein said anaerobic microbial fermentation is a production of said at least one organic compound from said cultivation medium and a gas comprising CO₂, CO or a mixture thereof.

31. The method according to item 30, wherein said gas is syngas, i.e. a mixture comprising CO and H₂.

32. The method according to any of the preceding items, wherein said cultivation medium comprises a nutrient selected from the group consisting of Al, Mn, Fe, Co, Zn, and Cu.

33. The method according to any of the preceding items, wherein said cultivation medium comprises Mn, Fe, Co, Zn and Cu.

34. The method according to any of the preceding items, wherein said cultivation medium comprises a nutrient selected from the group consisting of biotin, folic acid, pyridoxine, thiamine, riboflavin, nicotinic acid, D-pantothenate, vitamin B12, para-amino-benzoic acid and thiotic acid.

35. The method according to any of the preceding items, wherein said cultivation medium comprises biotin, folic acid, pyridoxine, thiamine, riboflavin, nicotinic acid, D-pantothenate, vitamin B12, para-amino-benzoic acid and thiotic acid.

36. The method according to any of the preceding items, wherein the growth efficiency is the specific growth rate.

37. The method according to any of the preceding items, wherein the growth efficiency is the biomass yield per carbon substrate provided in said cultivation medium.

38. The method according to any of the preceding items, wherein said microbial fermentation is a batch fermentation or a fed-batch fermentation.

39. The method according to any of the preceding items, wherein said microbial fermentation is a continuous fermentation.

40. The method according to any of the preceding items, wherein said microbial fermentation is an industrial scale process.

41. The method according to item 40, wherein said industrial scale process is a fermentation in at least 5 m³ scale, at least 30 m³ scale or at least 80 m³ scale.

42. A method for switching from a phase of mainly biomass growth to a phase of mainly product formation for an anaerobic microbial fermentation of a microorganism capable of converting CO₂, CO or a mixture thereof into at least one organic compound, wherein said product is said at least one organic compound and said method comprising growing the microorganisms in a cultivation medium wherein the concentrations of molybdenum and nickel remains substantially constant during the switch, and the concentrations of selenium and magnesium both increases 5-15 fold during the switch.

43. The method according to item 42, wherein the concentrations of selenium and magnesium both increases 7-13 fold during the switch.

44. The method according to any of items 42-43, wherein the concentration of selenium in the cultivation medium used before the switch is in the range from 2.0 μM to 8 μM, and the concentration of magnesium in the cultivation medium used before the switch is in the range from 0.15 mM to 0.30 mM.

45. The method according to any of items 42-44, wherein the concentration of molybdenum in the cultivation medium used before the switch is at least 1.5 M, and the concentration of nickel in the cultivation medium used before the switch is in the range from 3.0 μM to 8.5 μM.

46. The method according to any of items 42-45, wherein said phase of mainly biomass growth takes place with a cultivation medium wherein one or more hexoses is the main carbon source.

47. The method according to any of items 42-46, wherein said phase of mainly product formation takes place with a cultivation medium wherein a gas such as CO₂ or CO is the main carbon source.

48. The method according to any of items 42-47, wherein said phase of mainly biomass growth is conducted as a batch or fed-batch fermentation.

49. The method according to any of items 42-48, wherein said phase of mainly product formation is conducted as a fed-batch fermentation or a continuous fermentation.

50. The method according to any of items 42-49, wherein said switch of cultivation medium lasts less than 5 hours, less than 2 hours, less than 1 hour or less than 30 minutes.

51. Use of the method as defined in any of items 1-50 for the industrial manufacture of at least one organic compound from CO₂, CO or a mixture thereof.

52. The use according to item 51, wherein said at least one organic compound comprises a C₁₋₆-compound, such as a C₁₋₆-alcohol, a C₁₋₆-carboxylic acid or a C₁₋₆-ketone.

53. The use according to item 52, wherein said at least one organic compound comprises acetate.

54. The use according to any of items 51-53, wherein said at least one organic compound comprises ethanol.

### BRIEF DESCRIPTION OF THE FIGURES

FIG. 1. Significance of the compounds tested using Plackett-Burman design on the growth of Moorella thermoacetica on fructose. A t-value above 1.65 or below −1.65 indicates a significant effect at a 90% confidence level.

FIG. 2. Generated response surface plot showing the influence of yeast extract and MgCl₂ concentrations on biomass production after 24 hours of fermentation on fructose.

FIG. 3. Significance of the compounds tested using Plackett-Burman design on the growth of Moorella thermoacetica on fructose. A t-value above 1.65 or below −1.65 indicates a significant effect at a 90% confidence level.

FIG. 4. Generated response surface plots showing the influence of Na₂MoO₄, NiCl₂ and Na₂SeO₄ concentrations on biomass production after 24 hours of fermentation on fructose.

FIG. 5. Growth patterns of Moorella thermoacetica in the improved and original media using fructose as carbon source. The cell density is measured using a real-time monitoring device.

FIG. 6. Acetate concentrations achieved after 63 hours of fermentation with Moorella thermoacetica in the improved and original media using fructose as carbon source.

FIG. 7. Significance of the compounds tested using Plackett-Burman design on the growth of Moorella thermoacetica on CO₂. A t-value above 1.65 or below −1.65 indicates a significant effect at a 90% confidence level.

FIG. 8. Generated response surface plot showing the influence of MgCl₂ concentrations on biomass production after 24 hours of fermentation using CO₂ as carbon source.

FIG. 9. Significance of the compounds tested using Plackett-Burman design on the growth of Moorella thermoacetica on CO₂. A t-value above 1.65 or below −1.65 indicates a significant effect at a 90% confidence level.

FIG. 10. Generated response surface plot showing the influence of Na₂SeO₄ and Na₂MoO₄ concentrations on biomass production after 24 hours of fermentation using CO₂ as carbon source.

FIG. 11. Absorbance indicating biomass concentration in the improved and original media after 24 hours of fermentation using CO₂ as carbon source.

### DETAILED DESCRIPTION OF THE INVENTION

Unless specifically defined herein, all technical and scientific terms used have the same meaning as commonly understood by a skilled artisan in the fields of biotechnology, microbiology, fermentations and microbial growth and product formation.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of biotechnology, microbiology, fermentation technology which are within the skill of the art. Such techniques are explained fully in the literature.

### Cultivation Medium

In a first aspect the present invention provides a method for increasing the growth efficiency in an anaerobic microbial fermentation of a microorganism capable of converting CO₂, CO or a mixture thereof into at least one organic compound, the method comprising growing the microorganisms in a cultivation medium comprising selenium in a concentration which is at least 1.2 μM.

In a second aspect the present invention provides a method for increasing the growth efficiency in an anaerobic microbial fermentation of a microorganism converting CO₂, CO or a mixture thereof into at least one organic compound, the method comprising growing the microorganisms in a cultivation medium comprising nickel in a concentration which is in the range from 3.0 μM to 8.5 μM.

In a third aspect the present invention provides a method for increasing the growth efficiency in an anaerobic microbial fermentation of a microorganism converting CO₂, CO or a mixture thereof into at least one organic compound, the method comprising growing the microorganisms in a cultivation medium comprising molybdenum in a concentration which is at least 1.5 μM.

It is to be understood that in a cultivation medium the selenium concentration which is at least 1.2 μM, may be combined with nickel in a concentration which is in the range from 3.0 μM to 8.5 μM and with molybdenum in a concentration which is at least 1.5 μM. In fact, it is believed that this combination of these three metals is a preferred embodiment.

In an embodiment the cultivation medium comprises selenium in a concentration which is at least 1.3 μM, at least 2 μM, at least 3 μM, or at least 4 μM. In another embodiment the cultivation medium comprises selenium in a concentration which is in the range from 2.0 μM to 8 μM, in the range from 3.0 μM to 7 μM, in the range from 4 μM to 6 μM or in the range from 4.4 μM to 5.6 μM. In another embodiment the cultivation medium comprises selenium in a concentration which is in the range from 4.4 μM to 5.6 μM. In yet another embodiment the cultivation medium comprises selenium in a concentration which is at least 5.0 μM, at least 10 μM, at least 20 μM, or at least 30 μM. In yet another embodiment the cultivation medium comprises selenium in a concentration which is in the range from 5.0 μM to 70 μM, in the range from 10 μM to 60 μM, in the range from 20 μM to 50 μM or in the range from 30 μM to 50 μM.

In another embodiment the cultivation medium comprises nickel in a concentration which is in the range from 4.0 μM to 8.0 μM, in the range from 5.0 μM to 7.0 μM or in the range from 5.5 μM to 7.0 μM.

In another embodiment the cultivation medium comprises molybdenum in a concentration which is at least 1.7 μM, at least 2.0 μM, or at least 2.2 μM. In another embodiment the cultivation medium comprises molybdenum in a concentration which is in the range from 1.5 μM to 20 μM, in the range from 1.5 μM to 10 μM or in the range from 2.0 μM to 5 μM.

In another embodiment the concentration of magnesium in said cultivation medium is in the range from 0.05 mM to 40 mM. In another embodiment the microorganism grows on a hexose as the main carbon source and the concentration of magnesium in said cultivation medium is in the range from 0.05 mM to 1.3 mM, from 0.10 mM to 0.8 mM, or from 0.15 mM to 0.30 mM. In yet another embodiment the microorganism grows on a gas such as CO₂ or CO as the main carbon source and the concentration of magnesium in said cultivation medium is in the range from 10 mM to 40 mM, from 15 mM to 35 mM, or from 18 mM to 27 mM.

Different carbon sources may be used for propagating the microorganisms prior to the product formation phase, and such carbon sources may in some embodiments also be added during the product formation phase where CO₂, CO or a mixture thereof is converted into at least one organic compound.

In an embodiment the microorganism grows on a hexose as the main carbon source, such as fructose or glucose. In another embodiment the microorganism grows on CO₂, CO or a mixture thereof as the main carbon source.

Other useful medium components are apparent from the Examples and the items of the present application.

### Microorganisms

It is to be understood that a range of different microorganisms can be used in the present invention as long as they are capable of converting CO₂, CO or a mixture thereof into at least one organic compound. Typically useful microorganisms are anaerobic microorganisms.

In an embodiment the microorganism is an acetogen.

Different acetogens may be used in the present invention. In some embodiments the microorganism is capable of converting CO₂ into at least one organic compound. In some embodiment the microorganism is capable of converting CO into at least one organic compound.

In one embodiment the at least one organic compound is a C₁₋₆-compound.

In another embodiment the C₁₋₆-compound is a C₁₋₆-alcohol, a C₁₋₆-carboxylic acid or a C₁₋₆-ketone.

Other useful microorganisms are selected from the group consisting of Clostridium, Moorella, Thermoanaerobacter, Thermoanaerobacterium, Acetogenium, Acetobacterium, Acetoanaerobium, Butyribacterium, Eubacterium, Pyrococcus, Desulfobacterium and Carboxydothermus.

In an embodiment the microorganism is Moorella or Thermoanaerobacter.

In another embodiment the microorganism is Moorella thermoacetica or Moorella thermoautotrophica.

The microorganism used in the invention may be a naturally occurring microorganisms, it may be a microorganisms optimized through selection, and it may be a microorganism which is genetically engineered, e.g. for increased product formation or for rendering it capable of synthesizing a specific organic compound of interest.

In an embodiment the at least one organic compound comprises acetate. In another embodiment the at least one organic compound is mainly acetate.

In another embodiment the at least one organic compound comprises ethanol. In another embodiment the at least one organic compound is mainly ethanol.

Growth efficiency of a microorganism indicates how well the growth is under certain fermentation conditions. In one embodiment the growth efficiency is the specific growth rate. In another embodiment the growth efficiency is the biomass yield per carbon substrate provided in said cultivation medium.

### EXAMPLES

### Example 1

This experiment describes the growth improvement of Moorella thermoacetica on fructose, achieved by modifying the composition of the growth medium using a statistical approach. Initially, Plackett-Burman design is used to identify which components have a significant effect on cell growth. Then, response surface methodology (RSM) is used to determine improved concentrations of those components. The factors tested in this experiment are the following eight components: yeast extract, NH₄Cl, trace elements, vitamins, MgCl₂, CaCl₂, KH₂PO₄, and NaCl.

The following stock solutions are prepared and sterilised: fructose (180 g/L), yeast extract (100 g/L), NH₄Cl (40 g/L), MgCl₂·6H₂O (20 g/L), CaCl₂·2H₂O (10 g/L), KH₂PO₄ (20 g/L), NaCl (20 g/L), MES monohydrate (200 g/L, pH 6.5), cysteine-HCl (100 mM) and resazurin (0.01 g/L). The vitamin solution is filter-sterilised and contains biotin (2 mg/L), folic acid (2 mg/L), pyridoxine hydrochloride (10 mg/L), thiamine-HCl (5 mg/L), riboflavin (5 mg/L), nicotinic acid (5 mg/L), calcium-D-(+)-pantothenate (5 mg/L), Vitamin B12 (0.5 mg/L), p-aminobenzoic acid (5 mg/L), thioctic acid (5 mg/L). The trace element solution is prepared by dissolving nitrilotriacetic acid (2 g/L) in water while increasing the pH to 6.0 with 2 M KOH, after which MnSO₄·H₂O (1 g/L), Fe(SO₄)₂(NH₄)₂·6H₂O (0.8 g/L), CoCl₂·6H₂O (0.2 g/L), ZnSO₄·7H₂O (0.2 g/L), CuCl₂·2H₂O (20 mg/L), NiCl₂·6H₂O (20 mg/L), Na₂MoO₄·2H₂O (20 mg/L), Na₂SeO₄ (20 mg/L), Na₂WO₄·2H₂O (20 mg/L) are added.

Each of the eight components are screened on two levels (Table 1). The Placket-Burman matrix, describing the final concentration of each factor in the 12 experiments performed, is generated with the statistical program JMP (Table 2). The experimental procedure is performed in an anaerobic chamber to guarantee an anoxic environment. Anoxic and sterile stock solutions are mixed to reach final concentrations as indicated in Table 2, along with MES monohydrate (final concentration 20 g/L; pH 6.5), resazurin (0.001 g/L), fructose (10.8 g/L) and cysteine-HCl (1 mM). 4.9 mL of each medium mix is aliquoted to 3 wells of a 24-well deep-well plate, and 0.1 mL inoculum (OD₆₀₀=3.62) is added to all wells. The plates are placed in an anoxic box, flushed with N₂/CO₂ (80/20) and, with a final pressure of 0.8 bars, incubated at 60° C. without shaking. After 24 hours, the plates are removed from the incubator and the culture of each well is transferred to a 96-well microtiter plate. The absorbance is read in a spectrophotometer at 600 nm (Table 3).

TABLE 1
The two levels selected for each factor tested using
Plackett-Burman design and fructose as carbon source.

| | Yeast | | | | | | | |
|---|---|---|---|---|---|---|---|---|
| | extract | NH₄Cl | TE | Vitamins | MgCl₂•6H₂O | CaCl₂•2H₂O | KH₂PO₄ | NaCl |
| Level | (g/L) | (g/L) | (% v/v) | (% v/v) | (g/L) | (g/L) | (g/L) | (g/L) |
| High (+) | 2 | 1 | 2 | 2 | 0.5 | 0.1 | 0.5 | 1 |
| Low (−) | 0.5 | 0.4 | 1 | 1 | 0.1 | 0.05 | 0.1 | 0.1 |

TABLE 2
Plackett-Burman matrix used to examine which of the factors tested have a
significant effect on the growth of Moorella thermoacetica on fructose

| | | | | TE | | | | | |
|---|---|---|---|---|---|---|---|---|---|
| | | Yeast extract | NH₄Cl | (% | Vitamins | MgCl₂•6H₂O | CaCl₂•2H₂O | KH₂PO₄ | NaCl |
| Run | Pattern | (g/L) | (g/L) | v/v) | (% v/v) | (g/L) | (g/L) | (g/L) | (g/L) |
| 1 | ++++++++ | 2 | 1 | 2 | 2 | 0.5 | 0.1 | 0.5 | 1 |
| 2 | +−−−+−−+ | 2 | 0.4 | 1 | 1 | 0.5 | 0.05 | 0.1 | 1 |
| 3 | +−+++−−− | 2 | 0.4 | 2 | 2 | 0.5 | 0.05 | 0.1 | 0.1 |
| 4 | −−+−−+−+ | 0.5 | 0.4 | 2 | 1 | 0.1 | 0.1 | 0.1 | 1 |
| 5 | ++−−−+−− | 2 | 1 | 1 | 1 | 0.1 | 0.1 | 0.1 | 0.1 |
| 6 | −−+−+++− | 0.5 | 0.4 | 2 | 1 | 0.5 | 0.1 | 0.5 | 0.1 |
| 7 | −+++−−−+ | 0.5 | 1 | 2 | 2 | 0.1 | 0.05 | 0.1 | 1 |
| 8 | −−−+−−+− | 0.5 | 0.4 | 1 | 2 | 0.1 | 0.05 | 0.5 | 0.1 |
| 9 | +−−+−+++ | 2 | 0.4 | 1 | 2 | 0.1 | 0.1 | 0.5 | 1 |
| 10 | −+−+++−− | 0.5 | 1 | 1 | 2 | 0.5 | 0.1 | 0.1 | 0.1 |
| 11 | +++−−−+− | 2 | 1 | 2 | 1 | 0.1 | 0.05 | 0.5 | 0.1 |
| 12 | −+−−+−++ | 0.5 | 1 | 1 | 1 | 0.5 | 0.05 | 0.5 | 1 |

TABLE 3
Absorbance read-outs indicating biomass concentration after
24 hours of fermentation in the Plackett-Burman experiment

| | | Absorbance at 600 nm (a.u.) | | |
|---|---|---|---|---|
| Run | Pattern | Replicate 1 | Replicate 2 | Replicate 3 |
| 1 | ++++++++ | 0.825 | 0.720 | 0.784 |
| 2 | +−−−+−−+ | 0.839 | 0.842 | 0.806 |
| 3 | +−+++−−− | 0.891 | 0.769 | 0.702 |
| 4 | −−+−−+−+ | 0.750 | 0.776 | 0.772 |
| 5 | ++−−−+−− | 0.862 | 0.818 | 0.814 |
| 6 | −−+−+++− | 0.691 | 0.702 | 0.674 |
| 7 | −+++−−−+ | 0.818 | 0.742 | 0.688 |
| 8 | −−−+−−+− | 0.829 | 0.922 | 0.910 |
| 9 | +−−+−+++ | 0.864 | 0.809 | 0.878 |
| 10 | −+−+++−− | 0.711 | 0.692 | 0.705 |
| 11 | +++−−−+− | 0.824 | 0.839 | 0.887 |
| 12 | −+−−+−++ | 0.702 | 0.709 | 0.700 |

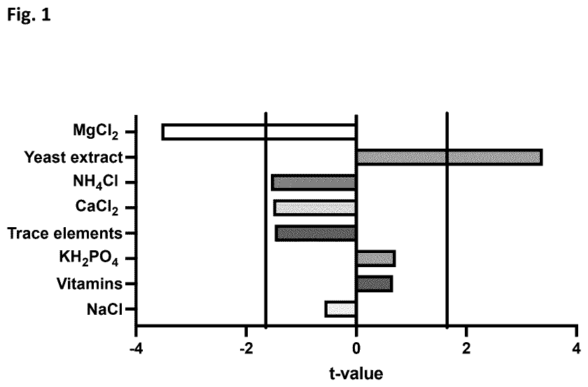

The results of the Plackett-Burman experiment are analysed with JMP (FIG. 1). Yeast extract and MgCl₂ are the only components with a significant effect on cell growth. MgCl₂ has a significantly negative effect on cell growth, while yeast extract has a significantly positive effect. Based on these results, a response surface methodology (RSM) experiment is performed to optimise the concentration of the two components.

The response surface experiment is performed as a central composite design with 2 centre points and an axial value of 1.414, and the respective matrix is generated by JMP (Table 4). The high and low values are chosen based on the Plackett-Burman experiment, and the centre and axial points are calculated by JMP. The axial and centre points are used to determine the curvature of the response surface. In the response matrix, −, +, 0, a and A represent the low value, high value, centre point, lower axial point and upper axial point, respectively. Yeast extract is shown to have a positive effect, so the low value for RSM is chosen as the high value in Plackett-Burman (2 g/L). The high value is arbitrarily chosen as 10 g/L. Conversely, MgCl₂·6H₂O has a negative effect, so the low value in Placket-Burman is chosen as the high value in the response surface experiment (0.1 g/L). The low value is chosen as 0.02 g/L.

TABLE 4
Response surface matrix used to optimise the concentrations
of yeast extract and MgCl₂ for growth of Moorella thermoacetica
on fructose

| Run | Pattern | Yeast extract (g/L) | MgCl₂•6H₂O (g/L) |
|---|---|---|---|
| 1 | a0 | 0.34 | 0.06 |
| 2 | −− | 2.00 | 0.02 |
| 3 | −+ | 2.00 | 0.10 |
| 4 | 0a | 6.00 | 0.003 |
| 5 | 00 | 6.00 | 0.06 |
| 6 | 00 | 6.00 | 0.06 |
| 7 | 0A | 6.00 | 0.12 |
| 8 | +− | 10.00 | 0.02 |
| 9 | ++ | 10.00 | 0.10 |
| 10 | A0 | 11.66 | 0.06 |

The entire procedure of the response surface experiment is performed in an anaerobic chamber. Ten medium solutions are prepared according to the response surface matrix (Table 4), where the concentration of yeast extract and MgCl₂·6H₂O is varied by adding the stock solutions to a medium containing MES monohydrate (final concentration 20 g/L; pH 6.5), NH₄Cl (0.4 g/L), KH₂PO₄ (0.5 g/L), trace elements (1% v/v), vitamins (1% v/v), CaCl₂·2H₂O (0.05 g/L), NaCl (0.4 g/L), fructose (5.4 g/L), resazurin (0.001 g/L) and cysteine-HCl (1 mM). 4.9 mL of each solution is aliquoted to 3 wells of a 24-well deep-well plate, and 0.1 ml inoculum (OD₆₀₀=0.343) is added to all wells. The plates are placed in an anoxic box flushed with N₂/CO₂ (80/20), with a final pressure of 0.8 bars. The box is incubated at 60° C. without shaking. After 24 hours, the plates are removed from the incubator and each solution is transferred to a 96-well microtiter plate. The absorbance is read in a spectrophotometer at 630 nm (Table 5).

TABLE 5
Absorbance read-outs indicating biomass concentration
of the RSM experiment on yeast extract and MgCl₂

Absorbance at 630 nm (a.u.)

| Run | Pattern | Replicate 1 | Replicate 2 | Replicate 3 |
|---|---|---|---|---|
| 1 | a0 | 0.518 | 0.607 | 0.466 |
| 2 | −− | 0.463 | 0.536 | 0.444 |
| 3 | −+ | 0.485 | 0.464 | 0.499 |
| 4 | 0a | 0.659 | 0.696 | 0.698 |
| 5 | 00 | 0.77 | 0.755 | 0.741 |
| 6 | 00 | 0.659 | 0.747 | 0.776 |
| 7 | 0A | 0.761 | 0.741 | 0.767 |
| 8 | +− | 0.883 | 0.896 | 0.909 |
| 9 | ++ | 0.727 | 0.708 | 0.703 |
| 10 | A0 | 0.751 | 0.735 | 0.759 |

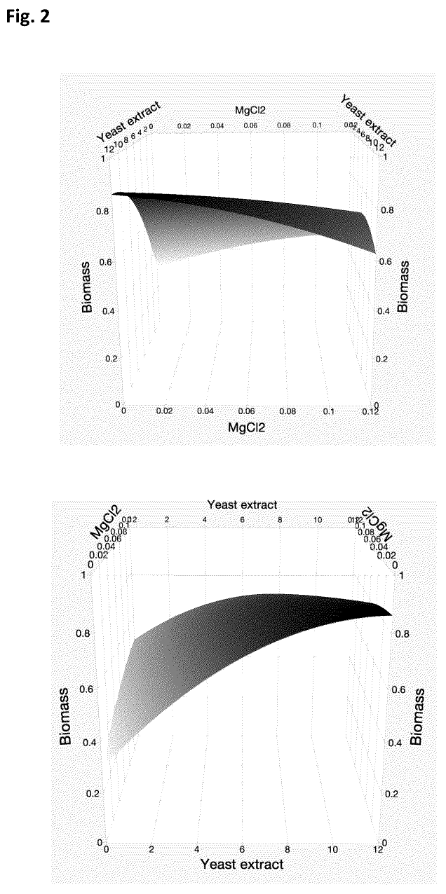

The results are analysed by JMP, which generates a second-degree polynomial equation (Equation 1) and the corresponding response surface plot (FIG. 2). The analysis shows that, when Moorella thermoacetica is grown on fructose, the biomass concentration increases with increased yeast extract and decreased MgCl₂ concentrations.

$$Second degree polynomial equation resulted from the RSM experiment on yeast extract and Mg {Cl}_{2}$$

$$0.738734065270186 ++ 0.119117490895689 * ( ( Yeast extract - 6 ) / 4 ) ++ - 0.00856188505625192 * ( ( {MgCl}_{2} - 0.06 ) / 0.04 ++ ( ( Yeast extract - 6 ) / 4 ) * ( ( ( {MgCl}_{2} - 0.06 ) / 0.04 ) * - 0.04625 ) ++ ( ( Yeast extract - 6 ) / 4 ) * ( ( ( Yeast extract - 6 ) / 4 ) * - 0.0595951861901537 ) ++ ( ( {MgCl}_{2} - 0.06 ) / 0.04 ) * ( ( ( {MgCl}_{2} - 0.06 ) / 0.04 ) * - 0.0161111425222695 ) . \qquad Equation 1$$

### Example 2

This experiment describes the statistical optimisation of selected trace elements for improved growth of Moorella thermoacetica on fructose. Plackett-Burman design is used to identify which components have a significant effect on cell growth and response surface methodology (RSM) is used to determine improved concentrations of those components. The factors tested in this experiment are the following 12 components: KAI(SO₄)₂, CoCl₂, CuCl₂, H₃BO₃, FeSO₄, MnSO₄, NiCl₂, nitrilotriacetic acid, Na₂SeO₄, Na₂MoO₄, Na₂WO₄ and ZnSO₄.

The following stock solutions are prepared and sterilised: fructose (500 g/L), yeast extract (100 g/L), NH₄Cl (40 g/L), MgCl₂·6H₂O (20 g/L), CaCl₂·2H₂O (10 g/L), KH₂PO₄ (20 g/L), NaCl (20 g/L), MES monohydrate (200 g/L; pH 6.5), cysteine-HCl (100 mM), resazurin (0.01 g/L), nitrilotriacetic acid (1 g/L; pH 6 adjusted with KOH), MnSO₄·H₂O (20 g/L), Fe(SO₄)·7H₂O (14 g/L), CoCl₂·6H₂O (10 g/L), ZnSO₄·7H₂O (4 g/L), KAI(SO₄)₂·12H₂O (1 g/L), CuCl₂·2H₂O (1 g/L), NiCl₂·6H₂O (1 g/L), Na₂MoO₄·2H₂O (1 g/L), Na₂SeO₄ (1 g/L) and Na₂WO₄·2H₂O (1 g/L). The vitamin solution is filter-sterilised and contains biotin (2 mg/L), folic acid (2 mg/L), pyridoxine hydrochloride (10 mg/L), thiamine-HCl (5 mg/L), riboflavin (5 mg/L), nicotinic acid (5 mg/L), calcium-D-(+)-pantothenate (5 mg/L), Vitamin B12 (0.5 mg/L), p-aminobenzoic acid (5 mg/L), thioctic acid (5 mg/L).

The initial screening is performed on two levels (Table 6). The Placket-Burman matrix, describing the final concentration of each factor in the 20 experiments performed, is generated with the statistical program JMP (Table 7). The experimental procedure is performed in an anaerobic chamber to guarantee an anoxic environment. Anoxic and sterile stock solutions are mixed to reach final concentrations as indicated in Table 7, along with MES monohydrate (final concentration 20 g/L; pH 6.5), fructose (5 g/L), yeast extract (0.5 g/L), NH₄Cl (0.4 g/L), MgCl₂·6H₂O (0.33 g/L), CaCl₂·2H₂O (0.05 g/L), KH₂PO₄ (0.5 g/L), NaCl (0.4 g/L), vitamin solution (1% v/v), resazurin (0.001 g/L) and cysteine-HCl (1 mM). 490 μL of each medium mix is aliquoted to 3 wells of a 96-well deep-well plate, and 10 μL inoculum (OD₆₀₀=0.6) is added to all wells. The plates are placed in an anoxic box, flushed with N₂/CO₂ (80/20) and, with a final pressure of 0.8 bars, incubated at 60° C. without shaking. After 24 hours, the plates are removed from the incubator and the culture of each well is transferred to a 96-well microtiter plate. The absorbance is read in a spectrophotometer at 630 nm (Table 8).

TABLE 6
The two levels selected for each factor tested using Plackett-Burman design and fructose as carbon source

| | KAI(SO₄)₂•12H₂O | CoCl₂•6H₂O | CuCl₂•2H₂O | H₃BO₃ | FeSO₄•7H₂O | MnSO₄•H₂O | Na₂MoO₄•2H₂O |
|---|---|---|---|---|---|---|---|
| Level | (mg/L) | (mg/L) | (mg/L) | (mg/L) | (mg/L) | (mg/L) | (mg/L) |
| High | 0.2 | 2 | 0.2 | 0.2 | 2.24 | 10 | 0.2 |
| (+) | | | | | | | |
| Low | 0.05 | 0.5 | 0.05 | 0.05 | 0.56 | 2.5 | 0.05 |
| (−) | | | | | | | |

| | NiCl₂•6H₂O | NTA* | Na₂SeO₄ | Na₂WO₄•2H₂O | ZnSO₄•7H₂O |
|---|---|---|---|---|---|
| Level | (mg/L) | (mg/L) | (mg/L) | (mg/L) | (mg/L) |
| High | 0.5 | 30 | 0.2 | 0.2 | 2 |
| (+) | | | | | |
| Low | 0.1 | 7.5 | 0.05 | 0.05 | 0.5 |
| (−) | | | | | |

*NTA = nitrilotriacetic acid

TABLE 7
Plackett-Burman matrix used to examine which of the factors tested have
a significant effect on the growth of Moorella thermoacetica on fructose

| | | KAI(SO₄)₂•12H₂O | CoCl₂•6H₂O | CuCl₂•2H₂O | H₃BO₃ | FeSO₄•7H₂O | MnSO₄•H₂O |
|---|---|---|---|---|---|---|---|
| Level | Pattern | (mg/L) | (mg/L) | (mg/L) | (mg/L) | (mg/L) | (mg/L) |
| 1 | −−−− | 0.05 | 0.5 | 0.05 | 0.05 | 2.24 | 10 |
| | ++−− | | | | | | |
| | +−−+ | | | | | | |
| 2 | −−−+ | 0.05 | 0.5 | 0.05 | 0.2 | 2.24 | 2.5 |
| | +−−+ | | | | | | |
| | −−++ | | | | | | |
| 3 | −−+− | 0.05 | 0.5 | 0.2 | 0.05 | 0.56 | 10 |
| | −+++ | | | | | | |
| | +−+− | | | | | | |
| 4 | −−++ | 0.05 | 0.5 | 0.2 | 0.2 | 0.56 | 2.5 |
| | −−+− | | | | | | |
| | −+++ | | | | | | |
| 5 | −−++ | 0.05 | 0.5 | 0.2 | 0.2 | 2.24 | 10 |
| | ++−+ | | | | | | |
| | −+−− | | | | | | |
| 6 | −+−− | 0.05 | 2 | 0.05 | 0.05 | 0.56 | 2.5 |
| | −−++ | | | | | | |
| | −−+− | | | | | | |
| 7 | −+−− | 0.05 | 2 | 0.05 | 0.05 | 2.24 | 10 |
| | ++++ | | | | | | |
| | −+−+ | | | | | | |
| 8 | −+−+ | 0.05 | 2 | 0.05 | 0.2 | 0.56 | 2.5 |
| | −−−− | | | | | | |
| | ++−− | | | | | | |
| 9 | −++− | 0.05 | 2 | 0.2 | 0.05 | 0.56 | 10 |
| | −+−− | | | | | | |
| | ++++ | | | | | | |
| 10 | −+++ | 0.05 | 2 | 0.2 | 0.2 | 2.24 | 2.5 |
| | +−+− | | | | | | |
| | +−−− | | | | | | |
| 11 | +−−− | 0.2 | 0.5 | 0.05 | 0.05 | 0.56 | 10 |
| | −++− | | | | | | |
| | −+−− | | | | | | |
| 12 | +−−+ | 0.2 | 0.5 | 0.05 | 0.2 | 0.56 | 2.5 |
| | −−++ | | | | | | |
| | ++−+ | | | | | | |
| 13 | +−−+ | 0.2 | 0.5 | 0.05 | 0.2 | 2.24 | 10 |
| | +++− | | | | | | |
| | +−+− | | | | | | |
| 14 | +−+− | 0.2 | 0.5 | 0.2 | 0.05 | 0.56 | 2.5 |
| | −−−+ | | | | | | |
| | +−−+ | | | | | | |
| 15 | +−+− | 0.2 | 0.5 | 0.2 | 0.05 | 2.24 | 2.5 |
| | +−−− | | | | | | |
| | −++− | | | | | | |
| 16 | ++−− | 0.2 | 2 | 0.05 | 0.05 | 2.24 | 2.5 |
| | +−−+ | | | | | | |
| | +++− | | | | | | |
| 17 | ++−+ | 0.2 | 2 | 0.05 | 0.2 | 0.56 | 10 |
| | −+−− | | | | | | |
| | −−++ | | | | | | |
| 18 | +++− | 0.2 | 2 | 0.2 | 0.05 | 2.24 | 2.5 |
| | +−+− | | | | | | |
| | −−−+ | | | | | | |
| 19 | ++++ | 0.2 | 2 | 0.2 | 0.2 | 0.56 | 10 |
| | −+−+ | | | | | | |
| | −−−− | | | | | | |
| 20 | ++++ | 0.2 | 2 | 0.2 | 0.2 | 2.24 | 10 |
| | ++++ | | | | | | |
| | ++++ | | | | | | |

| | Na₂MoO₄•2H₂O | NiCl₂•6H₂O | NTA* | Na₂SeO₄ | Na₂WO₄•2H₂O | ZnSO₄•7H₂O |
|---|---|---|---|---|---|---|
| Level | (mg/L) | (mg/L) | (mg/L) | (mg/L) | (mg/L) | (mg/L) |
| 1 | 0.05 | 0.1 | 30 | 0.05 | 0.05 | 2 |
| 2 | 0.05 | 0.5 | 7.5 | 0.05 | 0.2 | 2 |
| 3 | 0.2 | 0.5 | 30 | 0.05 | 0.2 | 0.5 |
| 4 | 0.2 | 0.1 | 7.5 | 0.2 | 0.2 | 2 |
| 5 | 0.05 | 0.5 | 7.5 | 0.2 | 0.05 | 0.5 |
| 6 | 0.2 | 0.5 | 7.5 | 0.05 | 0.2 | 0.5 |
| 7 | 0.2 | 0.5 | 7.5 | 0.2 | 0.05 | 2 |
| 8 | 0.05 | 0.1 | 30 | 0.2 | 0.05 | 0.5 |
| 9 | 0.05 | 0.1 | 30 | 0.2 | 0.2 | 2 |
| 10 | 0.2 | 0.1 | 30 | 0.05 | 0.05 | 0.5 |
| 11 | 0.2 | 0.1 | 7.5 | 0.2 | 0.05 | 0.5 |
| 12 | 0.2 | 0.5 | 30 | 0.2 | 0.05 | 2 |
| 13 | 0.2 | 0.1 | 30 | 0.05 | 0.2 | 0.5 |
| 14 | 0.05 | 0.5 | 30 | 0.05 | 0.05 | 2 |
| 15 | 0.05 | 0.1 | 7.5 | 0.2 | 0.2 | 0.5 |
| 16 | 0.05 | 0.5 | 30 | 0.2 | 0.2 | 0.5 |
| 17 | 0.05 | 0.1 | 7.5 | 0.05 | 0.2 | 2 |
| 18 | 0.2 | 0.1 | 7.5 | 0.05 | 0.05 | 2 |
| 19 | 0.05 | 0.5 | 7.5 | 0.05 | 0.05 | 0.5 |
| 20 | 0.2 | 0.5 | 30 | 0.2 | 0.2 | 2 |

*NTA = nitrilotriacetic acid

TABLE 8
Absorbance read-outs indicating biomass concentration after
24 hours of fermentation in the Plackett-Burman experiment

| | | Absorbance at 630 nm (a.u.) | | |
|---|---|---|---|---|
| Run | Pattern | Replicate 1 | Replicate 2 | Replicate 3 |
| 1 | −−−−++−−+−−+ | 0.395 | 0.53 | 0.554 |
| 2 | −−−++−−+−−++ | 0.572 | 0.575 | 0.569 |
| 3 | −−+−−++++−+− | 0.523 | 0.521 | 0.516 |
| 4 | −−++−−+−−+++ | 0.718 | 0.757 | 0.652 |
| 5 | −−++++−+−+−− | 0.603 | 0.645 | 0.666 |
| 6 | −+−−−−++−−+− | 0.594 | 0.59 | 0.607 |
| 7 | −+−−++++−+−+ | 0.941 | 0.921 | 0.908 |
| 8 | −+−+−−−−++−− | 0.795 | 0.752 | 0.76 |
| 9 | −++−−+−−++++ | 0.585 | 0.617 | 0.643 |
| 10 | −++++−+−+−−− | 0.498 | 0.491 | 0.511 |
| 11 | +−−−−++−−+−− | 0.733 | 0.744 | 0.738 |
| 12 | +−−+−−++++−+ | 0.737 | 0.733 | 0.723 |
| 13 | +−−++++−+−+− | 0.462 | 0.535 | 0.506 |
| 14 | +−+−−−−++−−+ | 0.506 | 0.457 | 0.472 |
| 15 | +−+−+−−−−++− | 0.623 | 0.61 | 0.636 |
| 16 | ++−−+−−++++− | 0.796 | 0.813 | 0.827 |
| 17 | ++−+−+−−−−++ | 0.463 | 0.503 | 0.437 |
| 18 | +++−+−+−−−−+ | 0.477 | 0.36 | 0.428 |
| 19 | ++++−+−+−−−− | 0.41 | 0.38 | 0.408 |
| 20 | ++++++++++++ | 0.674 | 0.67 | 0.7 |

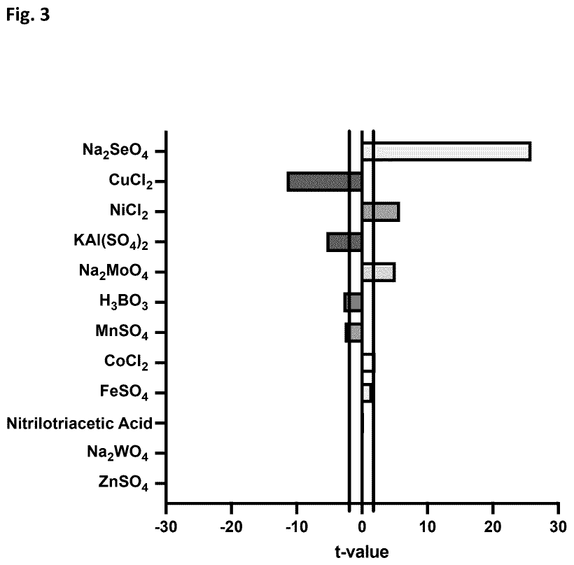

The results of the Plackett-Burman experiment are analysed with JMP (FIG. 3). Components containing selenium, nickel, molybdenum and cobalt have a significantly positive effect on cell growth, while copper, aluminium, boric acid and manganese have a significantly negative effect on cell growth. The five components with the strongest effect (Na₂SeO₄, CuCl₂, NiCl₂, KAI(SO₄)₂ and Na₂MoO₄) are further tested using response surface methodology, in order to optimise their concentration in the growth medium.

The response surface experiment is performed as a central composite design with 3 centre points and an axial value of 2 is chosen, and the respective matrix is generated by JMP (Table 9). The high and low values are chosen based on the Plackett-Burman experiment, and the centre and axial points are calculated by JMP. CuCl₂·2H₂O and KAI(SO₄)₂·12H₂O have a negative effect, so the high level in RSM is chosen as the low level in Plackett-Burman, namely 0.05 mg/L for both. The low level is arbitrarily chosen as 0.01 mg/L for both. Na₂MoO₄·2H₂O, NiCl₂·6H₂O and Na₂SeO₄ have a positive effect, so the low concentration in RSM is chosen as the high level in Plackett-Burman, as 0.2, 0.5 and 0.2 mg/L, respectively. The high levels are chosen arbitrarily as 0.6, 1.5 and 1 mg/L, respectively.

TABLE 9
Response surface matrix used to optimise the concentrations of KAI(SO₄)₂,
CuCl₂, Na₂MoO₄, NiCl₂ and Na₂SeO₄ for growth of Moorella thermoacetica on fructose

| | | KAI(SO₄)₂•12H₂O | CuCl₂•2H₂O | Na₂MoO₄•2H₂O | NiCl₂•6H₂O | Na₂SeO₄ |
|---|---|---|---|---|---|---|
| Run | Pattern | (mg/L) | (mg/L) | (mg/L) | (mg/L) | (mg/L) |
| 1 | −−−−− | 0.01 | 0.01 | 0.20 | 0.50 | 0.20 |
| 2 | −−−++ | 0.01 | 0.01 | 0.20 | 1.5 | 1.00 |
| 3 | −−+−+ | 0.01 | 0.01 | 0.6 | 0.50 | 1.00 |
| 4 | −−++− | 0.01 | 0.01 | 0.6 | 1.5 | 0.20 |
| 5 | −+−−+ | 0.01 | 0.05 | 0.2 | 0.5 | 1.00 |
| 6 | −+−+− | 0.01 | 0.05 | 0.20 | 1.50 | 0.20 |
| 7 | −++−− | 0.01 | 0.05 | 0.60 | 0.50 | 0.20 |
| 8 | −++++ | 0.01 | 0.05 | 0.60 | 1.5 | 1.00 |
| 9 | +−−−+ | 0.05 | 0.01 | 0.2 | 0.50 | 1.00 |
| 10 | +−−+− | 0.05 | 0.01 | 0.2 | 1.5 | 0.20 |
| 11 | +−+−− | 0.1 | 0.01 | 0.60 | 0.50 | 0.20 |
| 12 | +−+++ | 0.1 | 0.01 | 0.60 | 1.5 | 1.00 |
| 13 | ++−−− | 0.1 | 0.05 | 0.20 | 0.5 | 0.20 |
| 14 | ++−++ | 0.1 | 0.05 | 0.20 | 1.5 | 1.00 |
| 15 | +++−+ | 0.1 | 0.05 | 0.60 | 0.5 | 1.00 |
| 16 | ++++− | 0.1 | 0.05 | 0.60 | 1.5 | 0.20 |
| 17 | a0000 | 0.0 | 0.03 | 0.40 | 1.0 | 0.60 |
| 18 | A0000 | 0.1 | 0.03 | 0.40 | 1.0 | 0.60 |
| 19 | 0a000 | 0.0 | 0.00 | 0.40 | 1.0 | 0.60 |
| 20 | 0A000 | 0.0 | 0.07 | 0.40 | 1.0 | 0.60 |
| 21 | 00a00 | 0.0 | 0.03 | 0.00 | 1.0 | 0.60 |
| 22 | 00A00 | 0.0 | 0.03 | 0.8 | 1.00 | 0.60 |
| 23 | 000a0 | 0.0 | 0.03 | 0.4 | 0.00 | 0.60 |
| 24 | 000A0 | 0.0 | 0.03 | 0.40 | 2.00 | 0.60 |
| 25 | 0000a | 0.0 | 0.03 | 0.40 | 1.0 | 0.00 |
| 26 | 0000A | 0.0 | 0.03 | 0.4 | 1.00 | 1.40 |
| 27 | 00000 | 0.0 | 0.03 | 0.4 | 1.0 | 0.60 |
| 28 | 00000 | 0.0 | 0.03 | 0.4 | 1.0 | 0.60 |
| 29 | 00000 | 0.0 | 0.03 | 0.4 | 1.0 | 0.60 |

The entire procedure of the response surface experiment is performed in an anaerobic chamber. 29 medium solutions are prepared according to the response surface matrix (Table 9), where the concentrations of Na₂SeO₄, CuCl₂·2H₂O, NiCl₂·6H₂O, KAI(SO₄)₂·12H₂O and Na₂MoO₄·2H₂O are varied by adding the stock solutions to a medium containing MES monohydrate (final concentration 20 g/L; pH 6.5), NH₄Cl (0.4 g/L), KH₂PO₄ (0.5 g/L), CaCl₂·2H₂O (0.05 g/L), MgCl₂·6H₂O (0.33 g/L), NaCl (0.4 g/L), fructose (5 g/L), yeast extract (0.5 g/L), vitamins (1% v/v), CoCl₂·6H₂O (2 mg/L), H₃BO₃ (0.1 mg/L), FeSO₄·7H₂O (2.24 mg/L), MnSO₄·H₂O (10 mg/L), nitrilotriacetic acid (20 mg/L), Na₂WO₄·2H₂O (0.2 mg/L), ZnSO₄·7H₂O (2 mg/L), resazurin (0.001 g/L) and cysteine-HCl (1 mM). 490 μL of each solution is aliquoted to 3 wells of a 96-well deep-well plate, and 10 μL inoculum (OD₆₀₀=0.948) is added to all wells. The plates are placed in an anoxic box flushed with N₂/CO₂ (80/20), with a final pressure of 0.8 bars. The box is incubated at 60° C. without shaking. After 24 hours, the plates are removed from the incubator and each solution is transferred to a 96-well microtiter plate. The absorbance is read in a spectrophotometer at 630 nm (Table 10).

TABLE 10
Absorbance read-outs indicating biomass concentration of the RSM
experiment on KAl(SO₄)₂, CuCl₂, Na₂MoO₄, NiCl₂ and Na₂SeO₄

Absorbance at 630 nm (a.u.)

| Runs | Pattern | Replicate 1 | Replicate 2 | Replicate 3 |
|---|---|---|---|---|
| 1 | −−−−− | 1.165 | 1.19 | 1.2 |
| 2 | −−−++ | 1.176 | 1.234 | 1.222 |
| 3 | −−+−+ | 1.293 | 1.304 | 1.264 |
| 4 | −−++− | 1.212 | 1.171 | 1.194 |
| 5 | −+−−+ | 1.154 | 1.255 | 1.285 |
| 6 | −+−+− | 1.238 | 1.238 | 1.241 |
| 7 | −++−− | 1.223 | 1.238 | 1.2 |
| 8 | −++++ | 1.26 | 1.272 | 1.263 |
| 9 | +−−−+ | 1.162 | 1.26 | 1.312 |
| 10 | +−−+− | 1.263 | 1.272 | 1.261 |
| 11 | +−+−− | 1.277 | 1.237 | 1.197 |
| 12 | +−+++ | 1.288 | 1.274 | 1.254 |
| 13 | ++−−− | 1.131 | 1.203 | 1.234 |
| 14 | ++−++ | 1.262 | 1.285 | 1.271 |
| 15 | +++−+ | 1.288 | 1.299 | 1.305 |
| 16 | ++++− | 1.203 | 1.141 | 1.166 |
| 17 | a0000 | 1.148 | 1.265 | 1.313 |
| 18 | A0000 | 1.277 | 1.288 | 1.283 |
| 19 | 0a000 | 1.306 | 1.31 | 1.293 |
| 20 | 0A000 | 1.286 | 1.288 | 1.289 |
| 21 | 00a00 | 1.155 | 1.242 | 1.272 |
| 22 | 00A00 | 1.276 | 1.298 | 1.291 |
| 23 | 000a0 | 1.247 | 1.256 | 1.236 |
| 24 | 000A0 | 1.276 | 1.287 | 1.275 |
| 25 | 0000a | 0.317 | 0.504 | 0.474 |
| 26 | 0000A | 1.195 | 1.225 | 1.186 |
| 27 | 00000 | 1.239 | 1.226 | 1.211 |
| 28 | 00000 | 1.185 | 0.092 | 1.145 |
| 29 | 00000 | 1.095 | 1.095 | 1.102 |

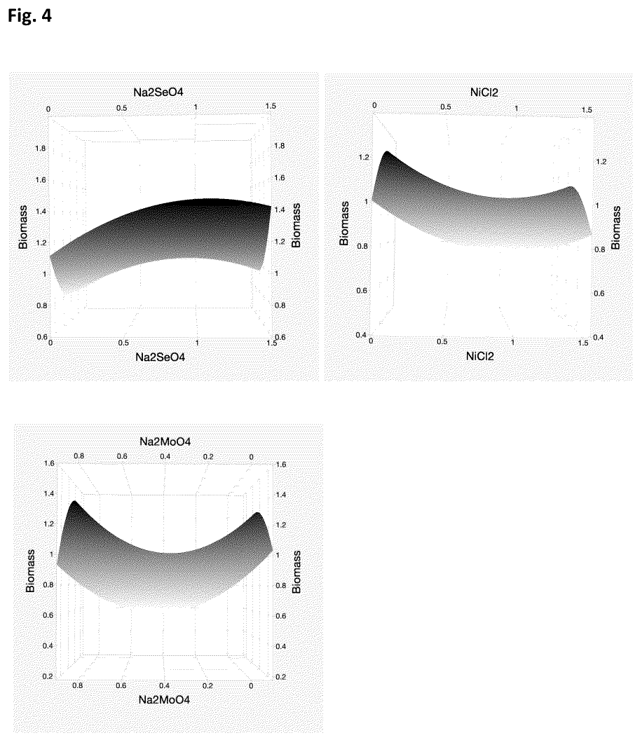

The results are analysed by JMP, which generates a second-degree polynomial equation (Equation 2) and the corresponding response surface plots (FIG. 4). The analysis shows that, within the boundaries tested, biomass concentration increases with increased Na₂SeO₄ concentration up to a critical level, after which it starts decreasing. At the same time, the highest biomass concentration is achieved at the highest Na₂MoO₄ concentration tested. With respect to NiCl₂, the model suggests that the highest biomass concentration is achieved when using a nickel concentration close to the two boundaries tested.

$$Second degree polynomial equation resulted from the RSM experiment on {KAl ( {SO}_{4} )}_{2} , {CuCl}_{2} , {Na}_{2} {MoO}_{4} , {NiCl}_{2} and {Na}_{2} {SeO}_{4}$$

$$1.01783470999122 + - 0.000929736362079477 * ( ( {KAl ( {SO}_{4} )}_{2} - 0.03 ) / 0.02 ) + - 0.0123591196301995 * ( ( {CuCl}_{2} - 0.03 ) / 0.02 ) + 0.00973611111111112 * ( ( {Na}_{2} {MoO}_{4} - 0.4 ) / 0.2 ) + 0.00254166666666666 * ( ( {NiCl}_{2} - 1 ) / 0.5 ) + 0.0833694065482988 * ( ( {Na}_{2} S e {O}_{4} - 0.6 ) / 0.4 ) + ( {KAl ( {SO}_{4} )}_{2} - 0.03 ) / 0.02 * ( {CuCl}_{2} - 0.03 ) / 0.02 * - 0.0106458333333333 + ( {KAl ( {SO}_{4} )}_{2} - 0.03 ) / 0.02 * ( {Na}_{2} {MoO}_{4} - 0.4 ) / 0.2 * - 0.00589583333333332 + ( {CuCl}_{2} - 0.03 ) / 0.02 * ( {Na}_{2} {MoO}_{4} - 0.4 ) / 0.2 * - 0.00389583333333333 + ( {KAl ( {SO}_{4} )}_{2} - 0.03 ) / 0.02 * ( {NiCl}_{2} - 1 ) / 0.5 * 0.00177083333333332 + ( {CuCl}_{2} - 0.03 ) / 0.02 * ( {NiCl}_{2} - 1 ) / 0.5 * 0.00135416666666668 + ( {Na}_{2} {MoO}_{4} - 0.4 ) / 0.2 * ( {NiCl}_{2} - 1 ) / 0.5 * - 0.0174791666666667 + ( {KAl ( {SO}_{4} )}_{2} - 0.03 ) / 0.02 * ( {Na}_{2} {SeO}_{4} - 0 \text{.6} ) / 0.4 * 0.00422916666666669 + ( {CuCl}_{2} - 0.03 ) / 0.02 * ( {Na}_{2} {SeO}_{4} - 0.6 ) / 0.4 * 0.00706249999999999 + ( {Na}_{2} {MoO}_{4} - 0 \text{.4} ) / 0.2 * ( {Na}_{2} {SeO}_{4} - 0.6 ) / 0.4 * 0.0138125 + ( {NiCl}_{2} - 1 ) / 0.5 * ( {Na}_{2} {SeO}_{4} - 0.6 ) / 0.4 * - 0.0046875 + ( {KAl ( {SO}_{4} )}_{2} - 0.03 ) / 0.02 * ( {KAl ( {SO}_{4} )}_{2} - 0.03 ) / 0.02 * 0.071733898175278 + ( {CuCl}_{2} - 0.03 ) / 0.02 * ( {CuCl}_{2} - 0.03 ) / 0.02 * 0.0817094897534409 + ( {Na}_{2} {MoO}_{4} - 0.4 ) / 0.2 * ( {Na}_{2} {MoO}_{4} - 0.4 ) / 0.2 * 0.0576734726195067 + ( {NiCl}_{2} - 1 ) / 0.5 * ( {NiCl}_{2} - 1 ) / 0.5 * 0.0594651392861734 + ( {Na}_{2} {SeO}_{4} - 0.6 ) / 0.4 * ( {Na}_{2} {SeO}_{4} - 0.6 ) / 0.4 * - 0.048660176726902 . \qquad Equation 2$$

### Example 3

This example describes the improved biomass production of Moorella thermoacetica in a growth medium containing fructose as carbon source and novel concentrations of certain nutrients.

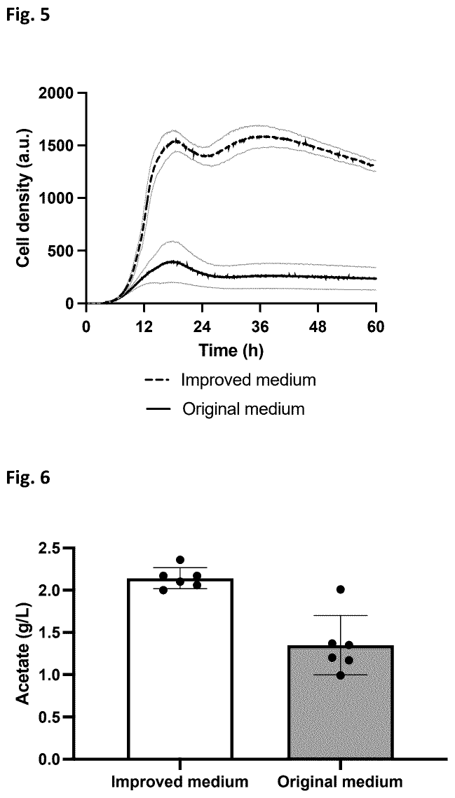

The procedure is performed in an anaerobic chamber to ensure an anoxic environment. As per Examples 1 and 2, sterile and anaerobic stock solutions of each component are prepared. Two media, termed “original” and “improved” media, are prepared by mixing stock solutions to reach the concentrations shown in Table 11. Then, 49 mL of each medium are aliquoted into three 125 mL serum bottles and inoculated with a growing culture of Moorella thermoacetica (OD₆₀₀=0.86) to reach an initial OD₆₀₀ of 0.02. The vials are incubated at 60° C. while stirring at 200 rpm using magnetic stirrers. The biomass concentration is monitored using a real-time cell density monitoring device. The growth pattern in the two media is presented in FIG. 5, which shows that the improved medium supports faster growth and higher biomass densities. According to end-point manual OD₆₀₀ measurements, biomass concentration is 63% higher in the improved medium than the original medium after 63 hours of fermentation. At the same time, FIG. 6 shows that the improved medium increased acetate production by 60% on average.

TABLE 11
Concentrations of components in the “original”
and “improved” media using fructose as carbon
source for fermentation with Moorella thermoacetica

| | Concentration in | Concentration in |
|---|---|---|
| Component | “original medium” | “improved medium” |
| Fructose (g/L) | 5 | 5 |
| MES monohydrate (g/L) | 20 | 20 |
| Resazurin (g/L) | 0.001 | 0.001 |
| Cysteine-HCl (mM) | 1 | 1 |
| Yeast extract (g/L) | 0.5 | 0.5 |
| NH₄Cl (g/L) | 0.4 | 0.4 |
| MgCl₂•6H₂O (g/L) | 0.33 | 0.05 |
| CaCl₂•2H₂O (g/L) | 0.05 | 0.05 |
| KH₂PO₄ (g/L) | 0.5 | 0.5 |
| NaCl (g/L) | 0.4 | 0.4 |
| Biotin (mg/L) | 0.02 | 0.12 |
| Folic acid (mg/L) | 0.02 | 0.02 |
| Pyridoxine-HCl (mg/L) | 0.1 | 0.1 |
| Thiamine-HCl (mg/L) | 0.05 | 0.1 |
| Riboflavin (mg/L) | 0.05 | 0.05 |
| Nicotinic acid (mg/L) | 0.05 | 0.05 |
| Ca-D-Pantothenate (mg/L) | 0.05 | 0.01 |
| Vit B12 (mg/L) | 0.005 | 0.0005 |
| p-aminobenzoic acid (mg/L) | 0.05 | 0.05 |
| Thioctic acid (mg/L) | 0.05 | 0.3 |
| CoCl₂•6H₂O (mg/L) | 2 | 2 |
| CuCl₂•2H₂O (mg/L) | 0.2 | 0.01 |
| FeSO₄•7H₂O (mg/L) | 5.6 | 5.6 |
| MnSO₄•H₂O (mg/L) | 10 | 7.5 |
| Na₂MoO₄•2H₂O (mg/L) | 0.2 | 0.6 |
| NiCl₂•6H₂O (mg/L) | 0.2 | 1.5 |
| Nitrilotriacetic acid (mg/L) | 20 | 20 |
| Na₂SeO₄ (mg/L) | 0.2 | 1 |
| Na₂WO₄•2H₂O (mg/L) | 0.2 | 0.2 |
| ZnSO₄•7H₂O (mg/L) | 2 | 2 |

### Example 4

This example describes the optimisation of medium composition with respect to five macronutrients specifically for growth on CO₂ and H₂ using Plackett-Burman design and response surface methodology.

The following stock solutions are prepared and sterilised: yeast extract (100 g/L), NH₄Cl (40 g/L), MgCl₂·6H₂O (20 g/L), CaCl₂·2H₂O (10 g/L), KH₂PO₄ (20 g/L), NaCl (20 g/L), MES monohydrate (200 g/L, pH 6.5), cysteine-HCl (100 mM) and resazurin (0.01 g/L). The trace elements solution contains nitrilotriacetic acid (1 g/L; pH 6 adjusted with KOH), MnSO₄·H₂O (20 g/L), Fe(SO₄)·7H₂O (14 g/L), CoCl₂·6H₂O (10 g/L), ZnSO₄·7H₂O (4 g/L), CuCl₂·2H₂O (1 g/L), NiCl₂·6H₂O (1 g/L), Na₂MoO₄·2H₂O (1 g/L), Na₂SeO₄ (1 g/L) and Na₂WO₄·2H₂O (1 g/L). The vitamin solution is filter-sterilised and contains biotin (2 mg/L), folic acid (2 mg/L), pyridoxine hydrochloride (10 mg/L), thiamine-HCl (5 mg/L), riboflavin (5 mg/L), nicotinic acid (5 mg/L), calcium-D-(+)-pantothenate (5 mg/L), Vitamin B12 (0.5 mg/L), p-aminobenzoic acid (5 mg/L), thioctic acid (5 mg/L).

Two levels are chosen for each factor tested (Table 12). The Placket-Burman matrix, describing the final concentration of each factor in the 20 experiments performed, is generated with the statistical program JMP (Table 13). The experimental procedure is performed in an anaerobic chamber to guarantee an anoxic environment. Anoxic and sterile stock solutions are mixed to reach final concentrations as indicated in Table 13, along with MES monohydrate (final concentration 20 g/L; pH 6.5), yeast extract (0.5 g/L), vitamin solution (1% v/v), trace elements solution (1% v/v) resazurin (0.001 g/L) and cysteine-HCl (1 mM). 490 μL of each medium mix is aliquoted to 3 wells of a 96-well deep-well plate, and 10 μL inoculum is added to all wells. The plates are placed in an anoxic box, flushed with H₂/CO₂ (80/20) for 10 minutes and, with a final pressure of 0.6 bars, incubated at 60° C. without shaking. After 24 hours, the plates are removed from the incubator and the culture of each well is transferred to a 96-well microtiter plate. The absorbance is read in a spectrophotometer at 630 nm (Table 14).

TABLE 12
The two levels selected for each factor tested using
Plackett-Burman design and CO₂ as carbon source

| | NH₄Cl | MgCl₂•6H₂O | CaCl₂•2H₂O | KH₂PO₄ | NaCl |
|---|---|---|---|---|---|
| Level | (g/L) | (g/L) | (g/L) | (g/L) | (g/L) |
| Low (−) | 0.2 | 0.05 | 0.01 | 0.1 | 0.1 |
| High (+) | 2 | 0.5 | 0.1 | 1 | 1 |

TABLE 13
Plackett-Burman matrix used to examine which of the factors tested have
a significant effect on the growth of Moorella thermoacetica on CO₂

| | | | MgCl₂•6H₂O | CaCl₂•2H₂O | | |
|---|---|---|---|---|---|---|
| Runs | Pattern | NH₄Cl (g/L) | (g/L) | (g/L) | KH₂PO₄ (g/L) | NaCl (g/L) |
| 1 | +++++ | 2.0 | 0.50 | 0.10 | 1.0 | 1.0 |
| 2 | −+−++ | 0.2 | 0.50 | 0.01 | 1.0 | 1.0 |
| 3 | −−+−+ | 0.2 | 0.05 | 0.10 | 0.1 | 1.0 |
| 4 | +−−+− | 2.0 | 0.05 | 0.01 | 1.0 | 0.1 |
| 5 | −+−−+ | 0.2 | 0.50 | 0.01 | 0.1 | 1.0 |
| 6 | −−+−− | 0.2 | 0.05 | 0.10 | 0.1 | 0.1 |
| 7 | −−−+− | 0.2 | 0.05 | 0.01 | 1.0 | 0.1 |
| 8 | +−−−+ | 2.0 | 0.05 | 0.01 | 0.1 | 1.0 |
| 9 | ++−−− | 2.0 | 0.50 | 0.01 | 0.1 | 0.1 |
| 10 | +++−− | 2.0 | 0.50 | 0.10 | 0.1 | 0.1 |
| 11 | −+++− | 0.2 | 0.50 | 0.10 | 1.0 | 0.1 |
| 12 | +−+++ | 2.0 | 0.05 | 0.10 | 1.0 | 1.0 |
| 13 | 00000 | 1.1 | 0.28 | 0.06 | 0.6 | 0.6 |
| 14 | 00000 | 1.1 | 0.28 | 0.06 | 0.6 | 0.6 |
| 15 | 00000 | 1.1 | 0.28 | 0.06 | 0.6 | 0.6 |

TABLE 14
Absorbance read-outs indicating biomass concentration after
24 hours of fermentation in the Plackett-Burman experiment

Absorbance at 630 nm (a.u.)

| Runs | Pattern | Replicate 1 | Replicate 2 | Replicate 3 |
|---|---|---|---|---|
| 1 | +++++ | 0.160 | 0.160 | 0.159 |
| 2 | −+−++ | 0.163 | 0.179 | 0.166 |
| 3 | −−+−+ | 0.169 | 0.168 | 0.165 |
| 4 | +−−+− | 0.138 | 0.150 | 0.140 |
| 5 | −+−−+ | 0.182 | 0.174 | 0.167 |
| 6 | −−+−− | 0.167 | 0.166 | 0.162 |
| 7 | −−−+− | 0.152 | 0.145 | 0.147 |
| 8 | +−−−+ | 0.136 | 0.138 | 0.136 |
| 9 | ++−−− | 0.164 | 0.159 | 0.154 |
| 10 | +++−− | 0.156 | 0.148 | 0.157 |
| 11 | −+++− | 0.157 | 0.150 | 0.149 |
| 12 | +−+++ | 0.133 | 0.134 | 0.134 |
| 13 | 00000 | 0.174 | 0.169 | 0.168 |
| 14 | 00000 | 0.164 | 0.161 | 0.157 |
| 15 | 00000 | 0.164 | 0.158 | 0.160 |

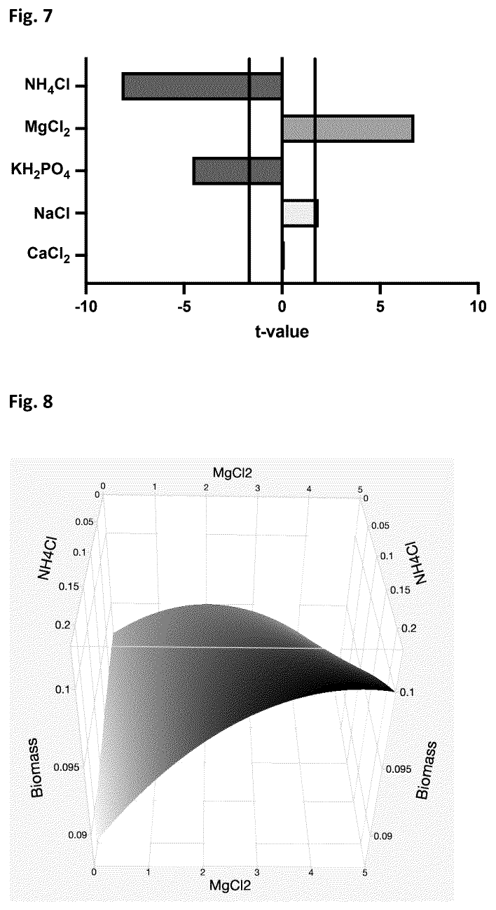

The results of the Plackett-Burman experiment are analysed with JMP (FIG. 7). The analysis shows that, when CO₂ is used as carbon source, NH₄Cl and KH₂PO₄ have a significantly negative effect on growth, while MgCl₂ and NaCl have a significantly positive effect. CaCl₂) does not have a significant effect. NH₄Cl, KH₂PO₄, MgCl₂ and NaCl are further tested using response surface methodology.

The response surface experiment is performed as a central composite design with 3 centre points and an axial value of 1 is chosen, and the respective matrix is generated by JMP (Table 15). The high and low values are chosen based on the Plackett-Burman experiment, and the centre and axial points are calculated by JMP. NH₄Cl and KH₂PO₄ have a negative effect in Plackett-Burman, so the high concentration in RSM is the same as the low concentration in Plackett-Burman, 0.2 and 0.1 g/L, respectively. The low value is chosen as 0.02 and 0.01 g/L, respectively. Since MgCl₂ and NaCl have a positive effect on growth, the low value in RSM is chosen as 0.5 and 1 g/L, respectively, and the high value as 5 g/L for both compounds.

TABLE 25
Response surface matrix used to optimise the concentrations
of NH₄Cl, MgCl₂, KH₂PO₄ and NaCl for growth of
Moorella thermoacetica on CO₂

| | | NH₄Cl | MgCl₂•6H₂O | KH₂PO₄ | NaCl |
|---|---|---|---|---|---|
| Runs | Pattern | (g/L) | (g/L) | (g/L) | (g/L) |
| 1 | −−−− | 0.02 | 0.50 | 0.01 | 1.0 |
| 2 | −−−+ | 0.02 | 0.50 | 0.01 | 5 |
| 3 | −−+− | 0.02 | 0.50 | 0.10 | 1 |
| 4 | −−++ | 0.02 | 0.50 | 0.10 | 5 |
| 5 | −+−− | 0.02 | 5.00 | 0.01 | 1 |
| 6 | −+−+ | 0.02 | 5.00 | 0.01 | 5 |
| 7 | −++− | 0.02 | 5.00 | 0.10 | 1 |
| 8 | −+++ | 0.02 | 5.00 | 0.10 | 5 |
| 9 | +−−− | 0.20 | 0.50 | 0.01 | 1 |
| 10 | +−−+ | 0.20 | 0.50 | 0.01 | 5 |
| 11 | +−+− | 0.20 | 0.50 | 0.10 | 1 |
| 12 | +−++ | 0.20 | 0.50 | 0.10 | 5 |
| 13 | ++−− | 0.20 | 5.00 | 0.01 | 1 |
| 14 | ++−+ | 0.20 | 5.00 | 0.01 | 5 |
| 15 | +++− | 0.20 | 5.00 | 0.10 | 1 |
| 16 | ++++ | 0.20 | 5.00 | 0.10 | 5 |
| 17 | a000 | 0.02 | 2.75 | 0.06 | 3 |
| 18 | A000 | 0.20 | 2.75 | 0.06 | 3 |
| 19 | 0a00 | 0.11 | 0.50 | 0.06 | 3 |
| 20 | 0A00 | 0.11 | 5.00 | 0.06 | 3 |
| 21 | 00a0 | 0.11 | 2.75 | 0.01 | 3 |
| 22 | 00A0 | 0.11 | 2.75 | 0.10 | 3 |
| 23 | 000a | 0.11 | 2.75 | 0.06 | 1 |
| 24 | 000A | 0.11 | 2.75 | 0.06 | 5 |
| 25 | 0000 | 0.11 | 2.75 | 0.06 | 3 |
| 26 | 0000 | 0.11 | 2.75 | 0.06 | 3 |
| 27 | 0000 | 0.11 | 2.75 | 0.06 | 3 |

The entire procedure of the response surface experiment is performed in an anaerobic chamber. 27 medium solutions are prepared according to the response surface matrix (Table 15), where the concentrations of NH₄Cl, KH₂PO₄, MgCl₂·6H₂O and NaCl are varied by adding the stock solutions to a medium containing MES monohydrate (final concentration 20 g/L; pH 6.5), CaCl₂·2H₂O (0.05 g/L), yeast extract (0.5 g/L), vitamins (1% v/v), trace elements solution (1% v/v), resazurin (0.001 g/L) and cysteine-HCl (1 mM). 490 μL of each solution is aliquoted to 3 wells of a 96-well deep-well plate, and 10 μL inoculum is added to all wells. The plates are placed in an anoxic box flushed with H₂/CO₂ (80/20) for 10 minutes, with a final pressure of 0.8 bars. The box is incubated at 60° C. without shaking. After 24 hours, the plates are removed from the incubator and each solution is transferred to a 96-well microtiter plate. The absorbance is read in a spectrophotometer at 630 nm (Table 16).

TABLE 16
Absorbance read-outs indicating biomass concentration
of the RSM experiment on NH₄Cl, MgCl₂, KH₂PO₄ and NaCl

Absorbance at 630 nm (a.u.)

| Runs | Pattern | Replicate 1 | Replicate 2 | Replicate 3 |
|---|---|---|---|---|
| 1 | −−−− | 0.104 | 0.105 | 0.098 |
| 2 | −−−+ | 0.085 | 0.089 | 0.089 |
| 3 | −−+− | 0.097 | 0.098 | 0.099 |
| 4 | −−++ | 0.089 | 0.084 | 0.084 |
| 5 | −+−− | 0.099 | 0.102 | 0.1 |
| 6 | −+−+ | 0.089 | 0.088 | 0.087 |
| 7 | −++− | 0.098 | 0.094 | 0.093 |
| 8 | −+++ | 0.085 | 0.085 | 0.083 |
| 9 | +−−− | 0.098 | 0.1 | 0.098 |
| 10 | +−−+ | 0.095 | 0.079 | 0.092 |
| 11 | +−+− | 0.092 | 0.088 | 0.101 |
| 12 | +−++ | 0.089 | 0.088 | 0.086 |
| 13 | ++−− | 0.116 | 0.115 | 0.114 |
| 14 | ++−+ | 0.106 | 0.099 | 0.101 |
| 15 | +++− | 0.096 | 0.097 | 0.096 |
| 16 | ++++ | 0.091 | 0.086 | 0.086 |
| 17 | a000 | 0.098 | 0.098 | 0.095 |
| 18 | A000 | 0.098 | 0.097 | 0.093 |
| 19 | 0a00 | 0.097 | 0.095 | 0.096 |
| 20 | 0A00 | 0.095 | 0.095 | 0.086 |
| 21 | 00a0 | 0.104 | 0.101 | 0.1 |
| 22 | 00A0 | 0.098 | 0.095 | 0.101 |
| 23 | 000a | 0.101 | 0.1 | 0.103 |
| 24 | 000A | 0.089 | 0.085 | 0.087 |
| 26 | 0000 | 0.096 | 0.099 | 0.095 |
| 27 | 0000 | 0.1 | 0.098 | 0.097 |

The results are analysed by JMP, which generates a second-degree polynomial equation (Equation 3). The corresponding response surface plot showing the MgCl₂ trend is presented in FIG. 7. When the carbon source is CO₂, biomass concentration increases with MgCl₂ concentration until a critical point is reached, after which the biomass concentration plateaus even when further increasing the MgCl₂ concentration.

$$Second degree polynomial equation resulted from the RSM experiment on {NH}_{4} Cl , {MgCl}_{2} , {KH}_{2} {PO}_{4} and NaCl$$

$$0.0969428571 \; 428572 + 0.00151851851851852 * ( ( {NH}_{4} Cl - 0.11 ) / 0.09 ) + 0.00124074074074074 * ( ( {MgCl}_{2} - 2.75 ) / 2.25 ) + - 0.0032222222 \; 2222222 * ( ( {KH}_{2} P {O}_{4} - 0.055 ) / 0.045 ) + - 0.00566666666666667 * ( ( NaCl - 3 ) / 2 ) + ( {NH}_{4} Cl - 0.11 ) / 0.09 * ( {MgCl}_{2} - 2.75 ) / 2.25 * 0.00239583333333333 + ( {NH}_{4} Cl - 0.11 ) / 0.09 * ( {KH}_{2} P {O}_{4} - 0.055 ) / 0.045 * - 0.00147916666666667 + ( {MgCl}_{2} - 2.75 ) / 2.2 {5}^{*} ( {KH}_{2} P {O}_{4} - 0.055 ) / 0.045 * - 0.00185416666666667 + ( {NH}_{4} Cl - 0.11 ) / 0.09 * ( NaCl - 3 ) / 2 * 0.000770833333333332 + ( {MgCl}_{2} - 2.75 ) / 2.25 * ( NaCl - 3 ) / 2 * - 0.000104166666666667 + ( {KH}_{2} P {O}_{4} - 0.055 ) / 0.045 * ( NaCl - 3 ) / 2 * 0.000770833333333333 + ( {NH}_{4} Cl - 0.11 ) / 0.09 * ( {NH}_{4} Cl - 0.11 ) / 0.09 * - 0.000257142857142861 + ( {MgCl}_{2} - 2.75 ) / 2.25 * ( {MgCl}_{2} - 2.75 ) / 2.25 * - 0.00275714285714286 + ( {KH}_{2} P {O}_{4} - 0.055 ) / 0.045 * ( {KH}_{2} {PO}_{4} - 0.055 ) / 0.045 * 0.00307619047619048 + ( NaCl - 3 ) / 2 * ( NaCl - 3 ) / 2 * - 0.00259047619047619 . \qquad Equation 3$$

### Example 5

This experiment describes the statistical optimisation of selected trace elements and vitamins for improved growth of Moorella thermoacetica on H₂/CO₂. Plackett-Burman design is used to identify which components have a significant effect on cell growth and response surface methodology (RSM) is used to determine improved concentrations of those components. The factors tested in this experiment are the following 14 components: biotin, thiamine-HCl, Ca-D-(+)-pantothenate, vitamin B12, nicotinic acid, thioctic acid, CoCl₂, CuCl₂, FeSO₄, MnSO₄, NiCl₂, Na₂SeO₄, Na₂WO₄, Na₂MoO₄.

The following stock solutions are prepared and sterilised: yeast extract (100 g/L), NH₄Cl (40 g/L), MgCl₂·6H₂O (20 g/L), CaCl₂·2H₂O (10 g/L), KH₂PO₄ (20 g/L), NaCl (20 g/L), MES monohydrate (200 g/L, pH 6.5), cysteine-HCl (100 mM), resazurin (0.01 g/L), nitrilotriacetic acid (1 g/L; pH 6 adjusted with KOH), MnSO₄·H₂O (20 g/L), Fe(SO₄)·7H₂O (14 g/L), CoCl₂·6H₂O (10 g/L), ZnSO₄·7H₂O (4 g/L), CuCl₂·2H₂O (1 g/L), NiCl₂·6H₂O (1 g/L), Na₂MoO₄·2H₂O (1 g/L), Na₂SeO₄ (1 g/L), Na₂WO₄·2H₂O (1 g/L), biotin (0.5 g/L), folic acid (1 g/L), pyridoxine hydrochloride (1 g/L), thiamine-HCl (1 g/L), riboflavin (0.1 g/L), nicotinic acid (1 g/L), calcium-D-(+)-pantothenate (1 g/L), Vitamin B12 (1 g/L), p-aminobenzoic acid (1 g/L) and thioctic acid (1 g/L).

The initial screening is performed on two levels (Table 17). The Placket-Burman matrix, describing the final concentration of each factor in the 23 experiments performed, is generated with the statistical program JMP (Table 18). The experimental procedure is performed in an anaerobic chamber to guarantee an anoxic environment. Anoxic and sterile stock solutions are mixed to reach final concentrations as indicated in Table 18, along with MES monohydrate (final concentration 20 g/L; pH 6.5), yeast extract (0.5 g/L), NH₄Cl (0.4 g/L), MgCl₂·6H₂O (0.33 g/L), CaCl₂·2H₂O (0.05 g/L), KH₂PO₄ (0.5 g/L), NaCl (0.4 g/L), folic acid (0.02 mg/L), pyridoxine-HCl (0.1 mg/L), riboflavin (0.05 mg/L), p-aminobenzoic acid (0.05 mg/L), ZnSO₄·7H₂O (2 mg/L), nitrilotriacetic acid (20 mg/L), resazurin (0.001 g/L) and cysteine-HCl (1 mM). 490 μL of each medium mix is aliquoted to 3 wells of a 96-well deep-well plate, and 10 μL inoculum is added to all wells. The plates are placed in an anoxic box, flushed with H₂/CO₂ (80/20) for 10 minutes and, with a final pressure of 0.8 bars, incubated at 60° C. without shaking. After 24 hours, the plates are removed from the incubator and the culture of each well is transferred to a 96-well microtiter plate. The absorbance is read in a spectrophotometer at 630 nm (Table 19).

TABLE 17
The two levels selected for each factor tested using Plackett-Burman design and CO₂ as carbon source

| | | | | Vitamin | Nicotinic | Thioctic | | |
|---|---|---|---|---|---|---|---|---|
| | Biotin | Thiamine-HCl | Ca-D-pantothenate | B12 | acid | acid | MnSO₄•H₂O | CuCl₂•2H₂O |
| Level | (mg/L) | (mg/L) | (mg/L) | (mg/L) | (mg/L) | (mg/L) | (mg/L) | (mg/L) |
| Low (−) | 0.02 | 0.05 | 0.05 | 0.005 | 0.05 | 0.05 | 5 | 0.05 |
| High (+) | 0.12 | 0.1 | 0.1 | 0.01 | 0.1 | 0.3 | 10 | 0.2 |

| | NiCl₂•6H₂O | Na₂MoO₄•2H₂O | Na₂SeO₄ | Na₂WO₄•2H₂O | CoCl₂•6H₂O | FeSO₄•7H₂O |
|---|---|---|---|---|---|---|
| Level | (mg/L) | (mg/L) | (mg/L) | (mg/L) | (mg/L) | (mg/L) |
| Low (−) | 0.2 | 0.2 | 0.2 | 0.2 | 2 | 5.6 |
| High (+) | 1.5 | 0.6 | 1 | 0.4 | 4 | 11.2 |

TABLE 18
Plackett-Burman matrix used to examine which of the factors tested have
a significant effect on the growth of Moorella thermoacetica on CO₂.

| | | | | | Vitamin | Nicotinic | Thioctic | | |
|---|---|---|---|---|---|---|---|---|---|
| | | Biotin | Thiamine-HCl | Ca-D-pantothenate | B12 | acid | acid | MnSO₄•H₂O | CuCl₂•2H₂O |
| Run | Pattern | (mg/L) | (mg/L) | (mg/L) | (mg/L) | (mg/L) | (mg/L) | (mg/L) | (mg/L) |
| 1 | −−−−+ | 0.02 | 0.05 | 0.05 | 0.005 | 0.10 | 0.30 | 5 | 0.05 |
| | +−−+− | | | | | | | | |
| | −+++ | | | | | | | | |
| 2 | −−−++ | 0.02 | 0.05 | 0.05 | 0.010 | 0.10 | 0.05 | 5 | 0.20 |
| | −−+−− | | | | | | | | |
| | ++++ | | | | | | | | |
| 3 | −−+−− | 0.02 | 0.05 | 0.10 | 0.005 | 0.05 | 0.30 | 10 | 0.20 |
| | ++++− | | | | | | | | |
| | +−+− | | | | | | | | |
| 4 | −−++− | 0.02 | 0.05 | 0.10 | 0.010 | 0.05 | 0.05 | 10 | 0.05 |
| | −+−−+ | | | | | | | | |
| | +++− | | | | | | | | |
| 5 | −−+++ | 0.02 | 0.05 | 0.10 | 0.010 | 0.10 | 0.30 | 5 | 0.20 |
| | +−+−+ | | | | | | | | |
| | −−−− | | | | | | | | |
| 6 | −+−−− | 0.02 | 0.10 | 0.05 | 0.005 | 0.05 | 0.05 | 10 | 0.20 |
| | −++−− | | | | | | | | |
| | +−−+ | | | | | | | | |
| 7 | −+−−+ | 0.02 | 0.10 | 0.05 | 0.005 | 0.10 | 0.30 | 10 | 0.20 |
| | +++−+ | | | | | | | | |
| | −+−− | | | | | | | | |
| 8 | −+−+− | 0.02 | 0.10 | 0.05 | 0.010 | 0.05 | 0.05 | 5 | 0.05 |
| | −−−++ | | | | | | | | |
| | −−+− | | | | | | | | |
| 9 | −++−− | 0.02 | 0.10 | 0.10 | 0.005 | 0.05 | 0.30 | 5 | 0.05 |
| | +−−++ | | | | | | | | |
| | ++−+ | | | | | | | | |
| 10 | −++++ | 0.02 | 0.10 | 0.10 | 0.010 | 0.10 | 0.05 | 10 | 0.05 |
| | −+−+− | | | | | | | | |
| | −−−+ | | | | | | | | |
| 11 | 00000 | 0.07 | 0.075 | 0.075 | 0.0075 | 0.075 | 0.175 | 7.5 | 0.125 |
| | 00000 | | | | | | | | |
| | 0000 | | | | | | | | |
| 12 | 00000 | 0.07 | 0.075 | 0.075 | 0.0075 | 0.075 | 0.175 | 7.5 | 0.125 |
| | 00000 | | | | | | | | |
| | 0000 | | | | | | | | |
| 13 | 00000 | 0.07 | 0.075 | 0.075 | 0.0075 | 0.075 | 0.175 | 7.5 | 0.125 |
| | 00000 | | | | | | | | |
| | 0000 | | | | | | | | |
| 14 | +−−−− | 0.12 | 0.05 | 0.05 | 0.005 | 0.05 | 0.30 | 10 | 0.05 |
| | ++−−+ | | | | | | | | |
| | −−++ | | | | | | | | |
| 15 | +−−+− | 0.12 | 0.05 | 0.05 | 0.010 | 0.05 | 0.05 | 10 | 0.20 |
| | −++++ | | | | | | | | |
| | −+−+ | | | | | | | | |
| 16 | +−−++ | 0.12 | 0.05 | 0.05 | 0.010 | 0.10 | 0.30 | 10 | 0.05 |
| | ++−+− | | | | | | | | |
| | +−−− | | | | | | | | |
| 17 | +−+−− | 0.12 | 0.05 | 0.10 | 0.005 | 0.05 | 0.05 | 5 | 0.20 |
| | −−++− | | | | | | | | |
| | −+−− | | | | | | | | |
| 18 | +−+−+ | 0.12 | 0.05 | 0.10 | 0.005 | 0.10 | 0.05 | 5 | 0.05 |
| | −−−−+ | | | | | | | | |
| | +−−+ | | | | | | | | |
| 19 | ++−−+ | 0.12 | 0.10 | 0.05 | 0.005 | 0.10 | 0.05 | 5 | 0.20 |
| | −−+++ | | | | | | | | |
| | +−+− | | | | | | | | |
| 20 | ++−+− | 0.12 | 0.10 | 0.05 | 0.010 | 0.05 | 0.30 | 5 | 0.05 |
| | +−−−− | | | | | | | | |
| | ++−− | | | | | | | | |
| 21 | +++−+ | 0.12 | 0.10 | 0.10 | 0.005 | 0.10 | 0.05 | 10 | 0.05 |
| | −+−−− | | | | | | | | |
| | −++− | | | | | | | | |
| 22 | ++++− | 0.12 | 0.10 | 0.10 | 0.010 | 0.05 | 0.30 | 5 | 0.20 |
| | +−+−− | | | | | | | | |
| | −−++ | | | | | | | | |
| 23 | +++++ | 0.12 | 0.10 | 0.10 | 0.010 | 0.10 | 0.30 | 10 | 0.20 |
| | +++++ | | | | | | | | |
| | ++++ | | | | | | | | |

| | NiCl₂•6H₂O | Na₂MoO₄•2H₂O | Na₂SeO₄ | Na₂WO₄•2H₂O | CoCl₂•6H₂O | FeSO₄•7H₂O |
|---|---|---|---|---|---|---|
| Run | (mg/L) | (mg/L) | (mg/L) | (mg/L) | (mg/L) | (mg/L) |
| 1 | 1.5 | 0.2 | 0.2 | 0.4 | 4 | 11.2 |
| 2 | 0.2 | 0.2 | 1.0 | 0.4 | 4 | 11.2 |
| 3 | 1.5 | 0.2 | 1.0 | 0.2 | 4 | 5.6 |
| 4 | 0.2 | 0.6 | 1.0 | 0.4 | 4 | 5.6 |
| 5 | 0.2 | 0.6 | 0.2 | 0.2 | 2 | 5.6 |
| 6 | 0.2 | 0.2 | 1.0 | 0.2 | 2 | 11.2 |
| 7 | 0.2 | 0.6 | 0.2 | 0.4 | 2 | 5.6 |
| 8 | 1.5 | 0.6 | 0.2 | 0.2 | 4 | 5.6 |
| 9 | 1.5 | 0.6 | 1.0 | 0.4 | 2 | 11.2 |
| 10 | 1.5 | 0.2 | 0.2 | 0.2 | 2 | 11.2 |
| 11 | 0.85 | 0.4 | 0.6 | 0.3 | 3 | 8.4 |
| 12 | 0.85 | 0.4 | 0.6 | 0.3 | 3 | 8.4 |
| 13 | 0.85 | 0.4 | 0.6 | 0.3 | 3 | 8.4 |
| 14 | 0.2 | 0.6 | 0.2 | 0.2 | 4 | 11.2 |
| 15 | 1.5 | 0.6 | 0.2 | 0.4 | 2 | 11.2 |
| 16 | 1.5 | 0.2 | 1.0 | 0.2 | 2 | 5.6 |
| 17 | 1.5 | 0.2 | 0.2 | 0.4 | 2 | 5.6 |
| 18 | 0.2 | 0.6 | 1.0 | 0.2 | 2 | 11.2 |
| 19 | 1.5 | 0.6 | 1.0 | 0.2 | 4 | 5.6 |
| 20 | 0.2 | 0.2 | 1.0 | 0.4 | 2 | 5.6 |
| 21 | 0.2 | 0.2 | 0.2 | 0.4 | 4 | 5.6 |
| 22 | 0.2 | 0.2 | 0.2 | 0.2 | 4 | 11.2 |
| 23 | 1.5 | 0.6 | 1.0 | 0.4 | 4 | 11.2 |

TABLE 19
Absorbance read-outs indicating biomass concentration after
24 hours of fermentation in the Plackett-Burman experiment

| | | Absorbance at 630 nm (a.u.) | | |
|---|---|---|---|---|
| Runs | Pattern | Replicate 1 | Replicate 2 | Replicate 3 |
| 1 | −−−−++−−+−−+++ | 0.13 | 0.126 | 0.121 |
| 2 | −−−++−−+−−++++ | 0.195 | 0.188 | 0.191 |
| 3 | −−+−−++++−+−+− | 0.184 | 0.178 | 0.179 |
| 4 | −−++−−+−−++++− | 0.21 | 0.198 | 0.207 |
| 5 | −−++++−+−+−−−− | 0.138 | 0.137 | 0.135 |
| 6 | −+−−−−++−−+−−+ | 0.188 | 0.187 | 0.183 |
| 7 | −+−−++++−+−+−− | 0.129 | 0.122 | 0.123 |
| 8 | −+−+−−−−++−−+− | 0.108 | 0.112 | 0.113 |
| 9 | −++−−+−−++++−+ | 0.222 | 0.215 | 0.221 |
| 10 | −++++−+−+−−−−+ | 0.128 | 0.121 | 0.121 |
| 11 | 00000000000000 | 0.175 | 0.163 | 0.165 |
| 12 | 00000000000000 | 0.157 | 0.163 | 0.166 |
| 13 | 00000000000000 | 0.185 | 0.181 | 0.18 |
| 14 | +−−−−++−−+−−++ | 0.126 | 0.123 | 0.119 |
| 15 | +−−+−−++++−+−+ | 0.117 | 0.113 | 0.12 |
| 16 | +−−++++−+−+−−− | 0.195 | 0.203 | 0.2 |
| 17 | +−+−−−−++−−+−− | 0.124 | 0.125 | 0.122 |
| 18 | +−+−+−−−−++−−+ | 0.223 | 0.213 | 0.212 |
| 19 | ++−−+−−++++−+− | 0.217 | 0.205 | 0.202 |
| 20 | ++−+−+−−−−++−− | 0.181 | 0.192 | 0.186 |
| 21 | +++−+−+−−−−++− | 0.131 | 0.124 | 0.122 |
| 22 | ++++−+−+−−−−++ | 0.112 | 0.109 | 0.111 |
| 23 | ++++++++++++++ | 0.205 | 0.195 | 0.196 |

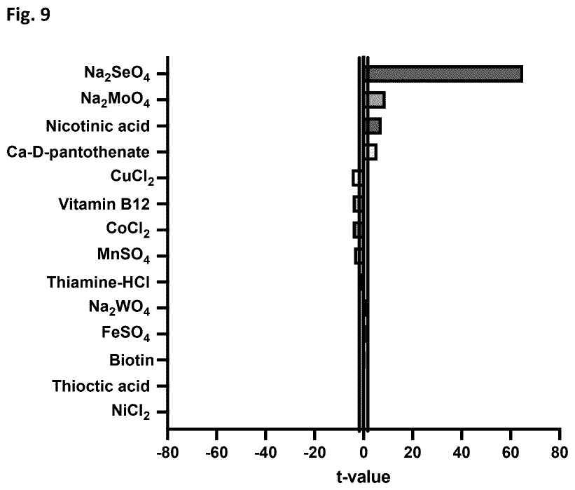

The results of the Plackett-Burman experiment are analysed with JMP (FIG. 9). Within the concentrations tested, components containing selenium, molybdenum, nicotinic acid, Ca-D-(+)-pantothenate and tungsten have a significantly positive effect on growth, while components containing copper, vitamin B12, cobalt, manganese and thiamine have a significantly negative effect. Several response surface experiments are performed based off these results, including an analysis on the four components Na₂SeO₄, Na₂MoO₄, nicotinic acid and Ca-D-(+)-pantothenate.

The response surface experiment is performed as a central composite design with 3 centre points and an axial value of 2 is chosen, and the respective matrix is generated by JMP (Table 20). The high and low values are chosen based on the Plackett-Burman experiment, and the centre and axial points are calculated by JMP. Since all factors have a positive effect in Plackett-Burman, the low levels in RSM are chosen as the high levels from Plackett-Burman, namely 1, 0.6, 0.1 and 0.1 mg/L for Na₂SeO₄, Na₂MoO₄·2H₂O, nicotinic acid and Ca-D-(+)-pantothenate, respectively. The high level is 10 times higher than the low level for each factor, resulting in 10, 6, 1 and 1 mg/L for Na₂SeO₄, Na₂MoO₄·2H₂O, nicotinic acid and Ca-D-(+)-pantothenate, respectively.

TABLE 20
Response surface matrix used to optimise the concentrations
of Na₂SeO₄, Na₂MoO₄, nicotinic acid and Ca-D-pantothenate for
growth of Moorella thermoacetica on CO₂

| | | Na₂SeO₄ | Na₂MoO₄•2H₂O | Nicotinic acid | Ca-D-pantothenate |
|---|---|---|---|---|---|
| Runs | Pattern | (mg/L) | (mg/L) | (mg/L) | (mg/L) |
| 1 | −−−− | 1 | 0.6 | 0.1 | 0.1 |
| 2 | −−−+ | 1 | 0.6 | 0.1 | 1.0 |
| 3 | −−+− | 1 | 0.6 | 1.0 | 0.1 |
| 4 | −−++ | 1 | 0.6 | 1.0 | 1.0 |
| 5 | −+−− | 1 | 6.0 | 0.1 | 0.1 |
| 6 | −+−+ | 1 | 6.0 | 0.1 | 1.0 |
| 7 | −++− | 1 | 6.0 | 1.0 | 0.1 |
| 8 | −+++ | 1 | 6.0 | 1.0 | 1.0 |
| 9 | +−−− | 10 | 0.6 | 0.1 | 0.1 |
| 10 | +−−+ | 10 | 0.6 | 0.1 | 1.0 |
| 11 | +−+− | 10 | 0.6 | 1.0 | 0.1 |
| 12 | +−++ | 10 | 0.6 | 1.0 | 1.0 |
| 13 | ++−− | 10 | 6.0 | 0.1 | 0.1 |
| 14 | ++−+ | 10 | 6.0 | 0.1 | 1.0 |
| 15 | +++− | 10 | 6.0 | 1.0 | 0.1 |
| 16 | ++++ | 10 | 6.0 | 1.0 | 1.0 |
| 17 | a000 | 0 | 3.3 | 0.55 | 0.55 |
| 18 | A000 | 14.5 | 3.3 | 0.55 | 0.55 |
| 19 | 0a00 | 5.5 | 0 | 0.55 | 0.55 |
| 20 | 0A00 | 5.5 | 8.7 | 0.55 | 0.55 |
| 21 | 00a0 | 5.5 | 3.3 | 0.00 | 0.55 |
| 22 | 00A0 | 5.5 | 3.3 | 1.45 | 0.55 |
| 23 | 000a | 5.5 | 3.3 | 0.55 | 0.00 |
| 24 | 000A | 5.5 | 3.3 | 0.55 | 1.45 |
| 25 | 0000 | 5.5 | 3.3 | 0.55 | 0.55 |
| 26 | 0000 | 5.5 | 3.3 | 0.55 | 0.55 |
| 27 | 0000 | 5.5 | 3.3 | 0.55 | 0.55 |

The entire procedure of the response surface experiment is performed in an anaerobic chamber. 27 medium solutions are prepared according to the response surface matrix (Table 20), where the concentrations of Na₂SeO₄, Na₂MoO₄·2H₂O, nicotinic acid and Ca-D-(+)-pantothenate are varied by adding the stock solutions to a medium containing MES monohydrate (final concentration 20 g/L; pH 6.5), NH₄Cl (0.4 g/L), KH₂PO₄ (0.5 g/L), CaCl₂·2H₂O (0.05 g/L), MgCl₂·6H₂O (0.33 g/L), NaCl (0.4 g/L), yeast extract (0.5 g/L), biotin (0.12 mg/L), folic acid (0.02 mg/L), pyridoxine-HCl (0.1 mg/L), thiamine-HCl (0.1 mg/L), riboflavin (0.05 mg/L), p-aminobenzoic acid (0.05 mg/L), thioctic acid (0.3 mg/L), vitamin B12 (0.0005 mg/L), KAI(SO₄)₂·12H₂O (0.01 mg/L), CoCl₂·6H₂O (2 mg/L), CuCl₂·2H₂O (0.01 mg/L), MnSO₄·H₂O (7.5 mg/L), FeSO₄·7H₂O (5.6 mg/L), nitrilotriacetic acid (20 mg/L), Na₂WO₄·2H₂O (0.2 mg/L), ZnSO₄·7H₂O (2 mg/L), resazurin (0.001 g/L) and cysteine-HCl (1 mM). 490 μL of each solution is aliquoted to 3 wells of a 96-well deep-well plate, and 10 μL inoculum is added to all wells. The plates are placed in an anoxic box flushed with H₂/CO₂ (80/20) for 10 minutes, with a final pressure of 0.8 bars. The box is incubated at 60° C. without shaking. After 24 hours, the plates are removed from the incubator and each solution is transferred to a 96-well microtiter plate. The absorbance is read in a spectrophotometer at 630 nm (Table 21).

TABLE 21
Absorbance read-outs indicating biomass concentration
of the RSM experiment on Na₂SeO₄, Na₂MoO₄,
nicotinic acid and Ca-D-pantothenate

Absorbance at 630 nm (a.u.)

| Runs | Pattern | Replicate 1 | Replicate 2 | Replicate 3 |
|---|---|---|---|---|
| 1 | −−−− | 0.128 | 0.12 | 0.117 |
| 2 | −−−+ | 0.116 | 0.114 | 0.113 |
| 3 | −−+− | 0.111 | 0.115 | 0.112 |
| 4 | −−++ | 0.11 | 0.11 | 0.11 |
| 5 | −+−− | 0.117 | 0.113 | 0.113 |
| 6 | −+−+ | 0.109 | 0.109 | 0.108 |
| 7 | −++− | 0.107 | 0.109 | 0.108 |
| 8 | −+++ | 0.106 | 0.105 | 0.107 |
| 9 | +−−− | 0.132 | 0.116 | 0.116 |
| 10 | +−−+ | 0.113 | 0.11 | 0.113 |
| 11 | +−+− | 0.111 | 0.11 | 0.109 |
| 12 | +−++ | 0.11 | 0.11 | 0.107 |
| 13 | ++−− | 0.13 | 0.119 | 0.121 |
| 14 | ++−+ | 0.118 | 0.114 | 0.116 |
| 15 | +++− | 0.116 | 0.116 | 0.113 |
| 16 | ++++ | 0.114 | 0.114 | 0.117 |
| 17 | a000 | 0.09 | 0.085 | 0.085 |
| 18 | A000 | 0.118 | 0.116 | 0.114 |
| 19 | 0a00 | 0.118 | 0.118 | 0.118 |
| 20 | 0A00 | 0.114 | 0.113 | 0.112 |
| 21 | 00a0 | 0.128 | 0.12 | 0.118 |
| 22 | 00A0 | 0.117 | 0.112 | 0.115 |
| 23 | 000a | 0.115 | 0.114 | 0.112 |
| 24 | 000A | 0.116 | 0.115 | 0.113 |
| 25 | 0000 | 0.127 | 0.12 | 0.118 |
| 26 | 0000 | 0.115 | 0.114 | 0.117 |
| 27 | 0000 | 0.117 | 0.117 | 0.119 |

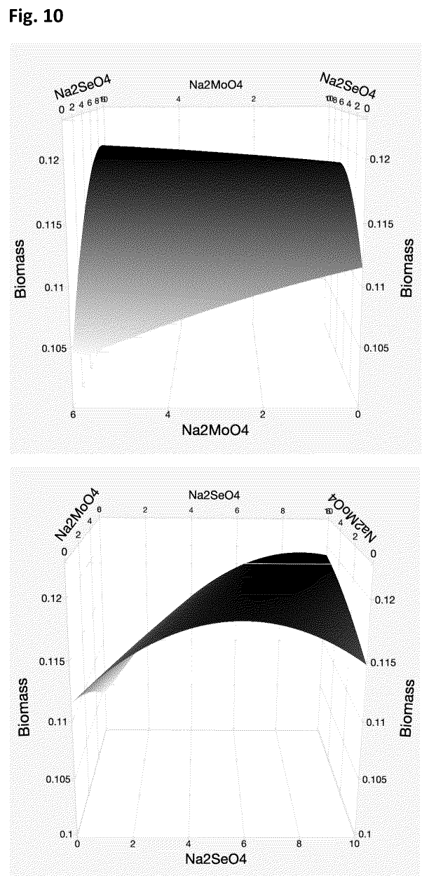

The results are analysed by JMP, which generates a second-degree polynomial equation (Equation 4) and the corresponding response surface plots (FIG. 10). The analysis shows that, within the concentrations tested and when CO₂ is used as carbon source, biomass concentration increases with decreased Na₂MoO₄. At the same time, biomass concentration increases with increased Na₂SeO₄ concentration until a critical level is reached, after which it starts to decline.

$$Second degree polynomial equation resulted from the RSM experiment on {Na}_{2} {SeO}_{4} , {Na}_{2} {MoO}_{4} , nicotinic acid and Ca - D - pantothenate$$

$$0.118222222222222 + 0.00352777777777778 * ( ( {Na}_{2} {SeO}_{4} - 5.5 ) / 4.5 ) + - 0.000611111111111111 * ( ( {Na}_{2} {MoO}_{4} - 3.3 ) / 2.7 ) + - 0.0 \; 0252777777777778 * ( ( Nicotinic acid - 0.55 ) / 0.45 + - 0.00138888888888889 * ( ( Ca - D - pantothenate - 0.55 ) / 0.45 ) + ( {Na}_{2} {SeO}_{4} - 5.5 ) / 4.5 * ( {Na}_{2} {MoO}_{4} - 3 \text{.3} ) / 2.7 * 0.00241666666666667 + ( {Na}_{2} {SeO}_{4} - 5.5 ) / 4.5 * ( Nicotinic acid - 0.55 ) / 0.45 * - 0.000083333333333332 + ( {Na}_{2} M o {O}_{4} - 3.3 ) / 2.7 * ( Nicotinic acid - 0.55 ) / 0.45 * 0.000583333333333334 + ( {Na}_{2} {SeO}_{4} - 5.5 ) / 4.5 * ( Ca - D - pantothenate - 0.55 ) / 0.45 * 8.10795832051072 e - 19 + ( {Na}_{2} M o {O}_{4} - 3.3 ) / 2.7 * ( Ca - D - pantothenate - 0.55 ) / 0.45 * 0.000333333333333333 + ( Nicotinic acid - 0.55 ) / 0.45 * ( Ca - D - pantothenate - 0.55 ) / 0.45 * 0.0015 + ( {Na}_{2} {SeO}_{4} - 5.5 ) / 4.5 * ( {Na}_{2} {SeO}_{4} - 5.5 ) / 4.5 * - 0.00401388888888889 + ( {Na}_{2} {MoO}_{4} - 3 \text{.3} ) / 2.7 * ( {Na}_{2} {MoO}_{4} - 3 \text{.3} ) / 2.7 * - 0.000472222222222224 + ( Nicotinic acid - 0.55 ) / 0.45 * ( Nicotinic acid - 0.55 ) / 0.45 * 0.00023611111111111 + ( Ca - D - pantothenate - 0.55 ) / 0.45 * ( Ca - D - pantothenate - 0.55 ) / 0.45 * - 0.000805555555555556 . \qquad Equation 4$$

### Example 6

This example describes the improved biomass production of Moorella thermoacetica in a growth medium containing novel concentrations of certain nutrients when CO₂ is used as carbon source.

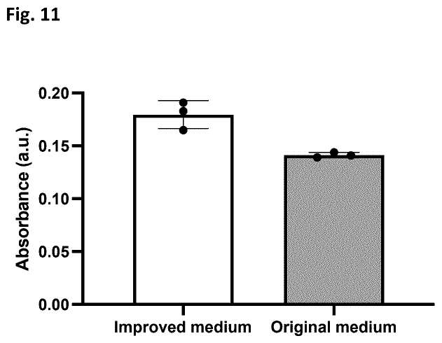

The procedure is performed in an anaerobic chamber to ensure an anoxic environment. As per Examples 4 and 5, sterile and anaerobic stock solutions of each component are prepared. Two media, termed “original” and “improved” media, are prepared by mixing stock solutions to reach the concentrations shown in Table 22. 490 μL of each solution is aliquoted to 3 wells of a 96-well deep-well plate, and 10 μL inoculum is added to all wells. The plates are placed in an anoxic box flushed with H₂/CO₂ (80/20) for 10 minutes, with a final pressure of 0.8 bars. The box is incubated at 60° C. without shaking. After 24 hours, the plates are removed from the incubator and each solution is transferred to a 96-well microtiter plate. The absorbance is read in a spectrophotometer at 630 nm (FIG. 11). Accordingly, the improved medium shows an average 27% increase in biomass concentration compared to the original medium.

TABLE 22
Concentrations of medium components in the “original”
and “improved” media using CO₂ as carbon
source for fermentation with Moorella thermoacetica

| | Concentration in | Concentration in |
|---|---|---|
| Component | “original medium” | “improved medium” |
| MES monohydrate (g/L) | 20 | 20 |
| Resazurin (g/L) | 0.001 | 0.001 |
| Cysteine-HCl (mM) | 1 | 1 |
| Yeast extract (g/L) | 0.5 | 0.5 |
| NH₄Cl (g/L) | 0.4 | 0.2 |
| MgCl₂•6H₂O (g/L) | 0.33 | 4.5 |
| CaCl₂•2H₂O (g/L) | 0.05 | 0.05 |
| KH₂PO₄ (g/L) | 0.5 | 0.01 |
| NaCl (g/L) | 0.4 | 1 |
| Biotin (mg/L) | 0.02 | 0.12 |
| Folic acid (mg/L) | 0.02 | 0.02 |
| Pyridoxine-HCl (mg/L) | 0.1 | 0.1 |
| Thiamine-HCl (mg/L) | 0.05 | 0.1 |
| Riboflavin (mg/L) | 0.05 | 0.05 |
| Nicotinic acid (mg/L) | 0.05 | 0.1 |
| Ca-D-Pantothenate (mg/L) | 0.05 | 0.1 |
| Vit B12 (mg/L) | 0.005 | 0.004 |
| p-aminobenzoic acid (mg/L) | 0.05 | 0.05 |
| Thioctic acid (mg/L) | 0.05 | 0.3 |
| CoCl₂•6H₂O (mg/L) | 2 | 0.2 |
| CuCl₂•2H₂O (mg/L) | 0.2 | 0.01 |
| FeSO₄•7H₂O (mg/L) | 5.6 | 5.6 |
| MnSO₄•H₂O (mg/L) | 10 | 3 |
| Na₂MoO₄•2H₂O (mg/L) | 0.2 | 0.6 |
| NiCl₂•6H₂O (mg/L) | 0.2 | 1.5 |
| Nitrilotriacetic acid (mg/L) | 20 | 20 |
| Na₂SeO₄ (mg/L) | 0.2 | 7.5 |
| Na₂WO₄•2H₂O (mg/L) | 0.2 | 0.2 |
| ZnSO₄•7H₂O (mg/L) | 2 | 2 |

1. A method for increasing the growth efficiency in an anaerobic microbial fermentation of a microorganism capable of converting CO₂, CO or a mixture thereof into at least one organic compound, the method comprising growing the microorganisms in a cultivation medium comprising selenium in a concentration which is at least 1.2 μM.

2. A method for increasing the growth efficiency in an anaerobic microbial fermentation of a microorganism converting CO₂, CO or a mixture thereof into at least one organic compound, the method comprising growing the microorganisms in a cultivation medium comprising nickel in a concentration which is in the range from 3.0 μM to 8.5 μM.

3. A method for increasing the growth efficiency in an anaerobic microbial fermentation of a microorganism converting CO₂, CO or a mixture thereof into at least one organic compound, the method comprising growing the microorganisms in a cultivation medium comprising molybdenum in a concentration which is at least 1.5 μM.

4. The method according to claim 1, wherein said cultivation medium comprises selenium in a concentration which is at least 1.3 μM, at least 2 μM, at least 3 μM, or at least 4 μM.

5. The method according to claim 1, wherein said cultivation medium comprises selenium in a concentration which is in the range from 2.0 μM to 8 μM, in the range from 3.0 μM to 7 μM, in the range from 4 μM to 6 μM or in the range from 4.4 μM to 5.6 μM.

6. The method according to claim 2, wherein said cultivation medium comprises nickel in a concentration which is in the range from 4.0 μM to 8.0 μM, in the range from 5.0 μM to 7.0 μM or in the range from 5.5 μM to 7.0 μM.

7. The method according to claim 3, wherein said cultivation medium comprises molybdenum in a concentration which is at least 1.7 μM, at least 2.0 μM, or at least 2.2 μM.

8. The method according to claim 1, wherein said microorganism is capable of converting CO₂ into at least one organic compound.

9. The method according to claim 1, wherein said microorganism is capable of converting CO into at least one organic compound.

10. The method according to claim 1, wherein said microorganism is an acetogen.

11. The method according to claim 10, wherein said microorganism is selected from Clostridium, Moorella, Thermoanaerobacter, Thermoanaerobacterium, Acetogenium, Acetobacterium, Acetoanaerobium, Butyribacterium, Eubacterium, Pyrococcus, Desulfobacterium and Carboxydothermus.

12. The method according to claim 1, wherein said at least one organic compound comprises acetate.

13. The method according to claim 1, wherein said anaerobic microbial fermentation is a production of said at least one organic compound from said cultivation medium and a gas comprising CO₂, CO or a mixture thereof.

14. Use of the method as defined in claim 1 for the industrial manufacture of at least one organic compound from CO₂, CO or a mixture thereof.

15. The use according to claim 14, wherein said at least one organic compound comprises acetate.

16. The method according to claim 3, wherein said microorganism is capable of converting CO₂ into at least one organic compound.

17. The method according to claim 3, wherein said microorganism is capable of converting CO into at least one organic compound.

18. The method according to claim 3, wherein said microorganism is an acetogen.

19. The method according to claim 18, wherein said microorganism is selected from Clostridium, Moorella, Thermoanaerobacter, Thermoanaerobacterium, Acetogenium, Acetobacterium, Acetoanaerobium, Butyribacterium, Eubacterium, Pyrococcus, Desulfobacterium and Carboxydothermus.

20. The method according to claim 3, wherein said at least one organic compound comprises acetate.
